SEQUENCE LISTING (57 sequences)

SEQ ID NO: 1 - DNA, 36 bp, Artificial Sequence
  Primer for PCR amplification of psbARP
aaaactgcagtcgactttcacagtttccattctgaa

SEQ ID NO: 2 - DNA, 27 bp, Artificial Sequence
  Primer for PCR amplification of psbA gene
catgccatggtaagatcttggtttatt

SEQ ID NO: 3 - DNA, 33 bp, Artificial Sequence
  Primer for introducing multiple cloning site into vector pVSR100
aattgagctcgaggtaccgcggtctagaagctt

SEQ ID NO: 4 - DNA, 33 bp, Artificial Sequence
  Primer for introducing multiple cloning site into vector pVSR100
aattaagcttctagaccgcggtacctcgagctc

SEQ ID NO: 5 - DNA, 42 bp, Artificial Sequence
  Primer for PCR amplification of psbA gene
attcgagctctaattaattaaggcttttctgctaacatatag

SEQ ID NO: 6 - DNA, 24 bp, Artificial Sequence
  Primer for PCR amplification of psbA gene
ggggtaccatcatttattggcaaa

SEQ ID NO: 7 - DNA, 27 bp, Artificial Sequence
  Primer for PCR amplification of 16SRP
ctggggtacctccccccgccacgatcg

SEQ ID NO: 8 - DNA, 42 bp, Artificial Sequence
  Primer for PCR amplification of 16SRP
cgcggatcctccctacaacttccaagcgcttcagattattag

SEQ ID NO: 9 - DNA, 30 bp, Artificial Sequence
  Primer for PCR amplification of rbcLRT
cgcggatccactaagtggataaaattagat

SEQ ID NO: 10 - DNA, 32 bp, Artificial Sequence
  Primer for PCR amplification of rbcLRT
gctctagattgtatttatttattgtattatac

SEQ ID NO: 11 - DNA, 42 bp, Artificial Sequence
  Primer for PCR amplification of aadA ge ne and 3′ end of rbcL gene
aaggtagttggcaaataactcgagactaagtggataaaatta

SEQ ID NO: 12 - DNA, 27 bp, Artificial Sequence
  Primer for PCR amplification of plastid targeting sequence
cccaagcttgaaagagataaattgaac

SEQ ID NO: 13 - DNA, 23 bp, Artificial Sequence
  Primer for PCR amplification of plastid targeting sequence
ccggaattctatctgaactactc

SEQ ID NO: 14 - DNA, 20 bp, Artificial Sequence
  Primer for primer extension analysis of aadA gene
cggccagcaacgtcggttcg

SEQ ID NO: 15 - DNA, 20 bp, Artificial Sequence
  Primer for primer extension analysis of vidA gene
atgcccacaggccgtcgagt

SEQ ID NO: 16 - DNA, 26 bp, Artificial Sequence
  Primer for primer extension analysis of rice and tobacco rrn promoter
ttcatagttgcattacttatagcttc

SEQ ID NO: 17 - DNA, 25 bp, Artificial Sequence
  Primer for PCR amplification of psbA
atgactgcaattttagagagacgcg

SEQ ID NO: 18 - DNA, 25 bp, Artificial Sequence
  Primer for PCR amplification of psbA
ttatccatttgtagatggagcttcg

SEQ ID NO: 19 - DNA, 20 bp, Artificial Sequence
  Primer for PCR amplification of 16SrRNA
cctggctcaggatgaacgct

SEQ ID NO: 20 - DNA, 22 bp, Artificial Sequence
  Primer for PCR amplification of 16SrRNA
ttcatgcaggcgagttgcagcc

SEQ ID NO: 21 - DNA, 34 bp, Artificial Sequence
  Primer for PCR amplification of fragment of psbA gene promoter
aaaactgcagtcgactagaaaggagcaatctgag

SEQ ID NO: 22 - DNA, 38 bp, Artificial Sequence
  Primer for PCR amplification of fragment of psbA gene promoter
aaaactgcagtcgacataaaaaaatttcactattctga

SEQ ID NO: 23 - DNA, 32 bp, Artificial Sequence
  Primer for PCR amplification of fragment of psbA gene promoter
catgccatggtaaaatcttggtttatttaatc

SEQ ID NO: 24 - DNA, 36 bp, Artificial Sequence
  Primer for PCR amplification of psbA gene promoter
aaaactgcagtcgacgcaacccactagcatatcgaa

SEQ ID NO: 25 - DNA, 480 bp, Oryza sativa
actttcacagtttccattctgaaatgttctctgtactataataaatagtaagtgaatcaacttactactaaaaaaattagtagacttcctcttcggaatagaaatagcctatttctacatagggaaagtcgtgtgcaatgaaaaatgcaagcacgatttggggagaggttttttctctattgtaacaaggaataattatctactccatccgactagttccgggttcgagtcccgggcaacccatatggaaactagaaaggagcaatctgagttttgatttttcactcacttcatttacaaaattttttggtttggtaaattttgttgtatggatatacaactgtcggggctggcttggttgacattggtatatagtctatattatactgttaaataacaagccttctattatctttctagttaatacgtgtgcttgggagtccttgcaatttaatttgaataaaccaagatcttaccatg

SEQ ID NO: 26 - DNA, 202 bp, Oryza sativa
actagaaaggagcaatctgagttttgatttttcactcacttcatttacaaaattttttggtttggtaaattttgttgtatggatatacaactgtcggggctggcttggttgacattggtatatagtctatattatactgttaaataacaagccttctattatctttctagttaatacgtgtgcttgggagtccttgcaattt

SEQ ID NO: 27 - DNA, 374 bp, Artificial Sequence
  3′ UTR of psbA gene
ggcttttctgctaacatatagcaatttttgaagaaaggaaagctagaaatacccaatatcttgctgaagcaagatattgggtatttcttttttttttattttgaatctttctattctgaattcagttaacgacgagatttagtatcctttcttgcactttcataactcgtgaaatgccgagttggtacgaattcccccaatttgcgacctaccataggatttgttatgtaaataggtatatgttcctttccattatgaatcgcgattgtatggccaaccattgcgggtagaatgctagatgcccgggaccacgttactattgtttctttctcctccttcatattgagcttttctatttttgccaataaatgatg

SEQ ID NO: 28 - DNA, 117 bp, Oryza sativa
ctccccccgccacgatcgaacgggaatggataagaggcttgtgggattgacgtgatagggtagggttggctatactgctggtggcgaactccaggctaataatctgaagcgcaagga

SEQ ID NO: 29 - DNA, 256 bp, Oryza sativa
actaagtggataaaattagatagaaaaaaggtctaaataaaaaagaagagaaatagaaagatcaaaaatcagttacgaaaatgcagtaattcttcttttttcttctaattgattgcaattaaactcgtctcaatctgaaaaaagattgagccgagtttaaatagattttgatacgatcatgagacttgacaaatcgggattcctctattctatatatttagaagatataaaggtataatacaataaataaatacaa

SEQ ID NO: 30 - DNA, 484 bp, Nicotiana tabacum
ataaaaaaatttcactattctgaaatgttgattgtaatagtaattaaggggtaaatcaactgagtattcaactttttaaagtctttctaatttctataagaaaggaactgatgtatacatagggaaagccgtgtgcaatgaaaaatgcaagcacggcttggggaggggtctttacttgtttatttaatttaagattaacatttattttatttaacaaggaacttatctactccatccgactagttccgggttcgaatcccgggcaacccactagcatatcgaaattctaattttctgtagagaagtccgtatttttccaatcaacttcattaaaaatttgaatagatctacatacaccttggttgacacgagtatataagtcatgttatactgttgaataaaaagccttccattttctattttgatttgtagaaaactagtgtgcttgggagtccctgatgattaaataaaccaagattttacc

SEQ ID NO: 31 - DNA, 222 bp, Nicotiana tabacum
gcaacccactagcatatcgaaattctaattttctgtagagaagtccgtatttttccaatcaacttcattaaaaatttgaatagatctacatacaccttggttgacacgagtatataagtcatgttatactgttgaataaaaagccttccattttctattttgatttgtagaaaactagtgtgcttgggagtccctgatgattaaataaaccaagattttacc

SEQ ID NO: 32 - DNA, 1261 bp, Oryza sativa
cctggctcaggatgaacgctggcggcatgcttaacacatgcaagtcggacgggaagtggtgtttccagtggcggacgggtgagtaacgcgtaagaacctgcccttgggaggggaacaacagctggaaacggctgctaataccccgtaggctgaggagcaaaaggaggaatccgcccgaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagctgacggagcaatgccgcgtggaggtagaaggcccacgggtcgtgaacttcttttcccggagaagaagcaatgacggtatctggggaataagcatcggctaactctgtgccagcagccgcggtaatacagaggatgcaagcgttatccggaatgattgggcgtaaagcgtctgtaggtggctttttaagtccgccgtcaaatcccagggctcaaccctggacaggcggtggaaactaccaagctggagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgaggtgagctaaccccaaaaacccgtcctcagttcggattgcaggctgcaactcgcctgcatgaa

SEQ ID NO: 33 - DNA, 1812 bp, Artificial Sequence
  PCR generated uidA coding region
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggcttttcgctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 34 - DNA, 795 bp, Artificial Sequence
  PCR generated aadA coding region
atggctcgtgaagcggttatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcactaaggtagttggcaaataa

SEQ ID NO: 35 - DNA, 2572 bp, Artificial Sequence
  PCR generated plastid targeting sequence
ttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggggtaatgcgccaggtgccgtagctaatcgagtagctctagaagcatgtgtaaaagctcgtaatgaaggacgtgatcttgctcaggaaggtaatgaaattattcgcgaggcttgcaaatggagcccggaactagctgctgcttgtgaagtatggaaagagatcgtatttaattttgcagcagtggacgttttggataagtaaaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctagaaaactaaatcaaaatctaagactcaaatctttctattgttgtcttggatccacaattaatcctacggatccttaggattggtatattcttttctatcctgtagtttgtagtttccctgaatcaagccaagtatcacacctctttctacccatcctgtatattgtcccctttgttccgtgttgaaatagaaccttaatttattacttatttttttattaaattttagatttgttagtgattagatattagtattagacgagattttacgaaacaattatttttttatttctttataggagaggacaaatctcttttttcgatgcgaatttgacacgacataggagaagccgccctttattaaaaattatattattttaaataatataaagggggttccaacatattaatatatagtgaagtgttcccccagattcagaactttttttcaatactcacaatccttattagttaataatcctagtgattggatttctatgcttagtctgataggaaataagatattcaaataaataattttatagcgaatgactattcatctattgtattttcatgcaaatagggggcaagaaaactctatggaaagatggtggtttaattcgatgttgtttaagaaggagttcgaacgcaggtgtgggctaaataaatcaatgggcagtcttggtcatattgaaaataccaatgaagatccaaatagaaaagtgaaaaacattcatagttggaggaatagtgacaattctagttgcagtaatgttgattatttattcggcgttaaagacattcggaatttcatctctgatgacacttttttagttagtgataggaatggagacagttattccatatattttgatattgaaaatcatatttttgagattgacaacgatcattcttttctgagtgaactagaaagttctttttatagttatcgaaactcgaattataggaataatggatttaggggcgaagatccctactataattcttacatgtatgatactcaatatagttggaataatcacattaatagttgcattgatagttatcttcagtctcaaatctgtatagatacttccattataagtggtagtgagaattacggtgacagttacatttatagggccgtttgtggtggtgaaagtcgaaatagtagtgaaaacgagggttccagtagacgaactcgcacgaagggcagtgatttaactataagagaaagttctaatgatctcgaggtaactcaaaaatacaggcatttgtgggttcaatgcgaaaattgttatggattaaattataagaaatttttgaaatcaaaaatgaatatttgtgaacaatgtggatatcatttgaaaatgagtagttcagatagaatt

SEQ ID NO: 36 - DNA, 28 bp, Artificial Sequence
  Primer for PCR amplification of 3′ end of psbA gene
attcgagctcttatttcaatgatattat

SEQ ID NO: 37 - DNA, 28 bp, Artificial Sequence
  Primer for PCR amplification of 3′ end of psbA gene
ggggtaccgatcctggcctagtctatag

SEQ ID NO: 38 - DNA, 375 bp, Oryza sativa
tcttatttcaatgatattattatttcaaagataagagatattcaaagataagagataagaagaagtcaaaatttgattttttttttggaaaaaaaaaatcaaaaagatatagtaacattagcaagaagagaaacaagttctatttcacaatttaaacaaatacaaaatcaaaatagaatactcaatcatgaataaatgcaagaaaataacctctccttatttttctataatgtaaacaaaaaagtatatgtaagtaaaatactagtaaataaataaaaagaaaaaaagaaaggagcaatagcacactattgatagaacaagaaaatgattattgatcatttcttttcaaaacctcctatagactaggccaggatc

SEQ ID NO: 39 - DNA, 1062 bp, Oryza sativa
ttatccatttgtagatggagcttcgatagcagataggtatagagggaagttgtgagcattacgttcatgcataacttccataccaaggttagcacggttaatgatatcagcccaagtattaattacacggccttgactgtcaactacagattggttgaaattgaaaccatttaggttgaaagccatagtgctgatacctaaagcggtaaaccagatacctactacaggccaagcagctaggaagaagtgtaacgaacgagagttgttgaaactagcatattggaagatcaatcggccaaaataaccatgagcggctacgatgttataagtttcttcctcttgaccgaatctgtaaccttcattagcagattcattttctgtggtttccctgatcaaactagaagttaccaaggaaccatgcatagcactgaatagggagccgccgaatacaccagctacgcctaacatgtgaaatgggtgcataaggatgttgtgctcagcctggaatacaatcatgaaattgaaagtaccagagattcctagaggcataccatcagaaaaacttccttgaccaattgggtagatcaagaaaactgcggtagcagctgcaacaggagctgaatatgcaacagcaatccaaggtcgcatacccagacggaaactaagctcccactcacgacccatgtaacaagctacgccaagtaagaagtgtagaacaattagttcataaggaccaccgttgtataaccattcatgaacggatgccgcttcccagattgggtaaaaatgtaaacctatagctgcagaagtaggaataatggcaccggaaataatattgtttccgtaaagtagagaccctgaaacaggttcacgaataccatcaatgtctactggaggagcagcaatgaaggcaataataaatacagaagttgccgtcaataaggtagggatcatcaaaacaccaaaccatccaatgtaaagacggttttcagtgctagttatccagttacagaagcgaccccataggctttcgctttcgcgtctctctaaaattgcagtcat

SEQ ID NO: 40 - DNA, 10 bp, Artificial Sequence
  Cla I site linker
gatcatcgat

SEQ ID NO: 41 - DNA, 32 bp, Artificial Sequence
  Primer for PCR amplification of uidA
cgcggatccatggtccgtcctgtagaaacccc

SEQ ID NO: 42 - DNA, 36 bp, Artificial Sequence
  Primer for PCR amplification of uidA
gctcgagctcccgggtcattgtttgcctccctgctg

SEQ ID NO: 43 - DNA, 31 bp, Artificial Sequence
  Primer for PCR amplification of aadA
cgcggatcctatggctcgtgaagcggttatc

SEQ ID NO: 44 - DNA, 32 bp, Artificial Sequence
  Primer for PCR amplification of aadA
ccgctcgagttatttgccaactaccttagtga

SEQ ID NO: 45 - DNA, 38 bp, Artificial Sequence
  Primer for inserting MCS vector to generate p326IFNG1
gatcttaccatgggcccgcggaggcctatcgatgagct

SEQ ID NO: 46 - DNA, 30 bp, Artificial Sequence
  Primer for inserting MCS vector to generate p326IFNG1
catgcataggcctccgcgggcccatggtaa

SEQ ID NO: 47 - DNA, 36 bp, Artificial Sequence
  Primer for inserting 6x His-tag to generate vector pVSRIFNG2
catgccatgggacacgtgcatcaccatcaccatcac

SEQ ID NO: 48 - DNA, 29 bp, Artificial Sequence
  Primer for inserting 6xHis-tag to generate vector pVSRIFNG2
ccgcgggccccgtgatggtgatggtgatg

SEQ ID NO: 49 - DNA, 44 bp, Artificial Sequence
  Primer for PCR amplification of ifnG gene
ggccggggcccatcgaaggtcgccaggacccgtacgttaaagaa

SEQ ID NO: 50 - DNA, 33 bp, Artificial Sequence
  Primer for PCR amplification of ifnG gene
cgccgggcccttactgagaagcacgacgaccgc

SEQ ID NO: 51 - DNA, 32 bp, Artificial Sequence
  Primer for PCR amplification of uidA gene
cgcggatccatgttacgtcctgtagaaacccc

SEQ ID NO: 52 - DNA, 35 bp, Artificial Sequence
  Primer for PCR amplification of uidA gene
ggggtacccttagattgtttgcctcccctgctgcg

SEQ ID NO: 53 - DNA, 42 bp, Artificial Sequence
  Primer for PCR amplification of ifnG gene
aaactgcagatcgaaggtcgccaggacccgtacgttaaagaa

SEQ ID NO: 54 - DNA, 31 bp, Artificial Sequence
  Primer for PCR amplification of ifnG gene
ctgcatgcatctctagactattactgagaag

SEQ ID NO: 55 - DNA, 30 bp, Artificial Sequence
  Primer for PCR amplification of uidAinfG fusion gene
tgctctagactattactgagaagcacgacg

SEQ ID NO: 56 - DNA, 30 bp, Artificial Sequence
  Primer for PCR amplification of ifnG
tgctctagatgcaggacccgtacgttaaag

SEQ ID NO: 57 - DNA, 30 bp, Artificial Sequence; identical to SEQ ID NO: 55
  Primer for PCR amplification of infG

(19) United States
(12) Patent Application Publication
(10) Pub. No.: US 2004/0253586 A1
(43) Pub. Date: Dec. 16, 2004

(54) Plastid transformation

(76) Inventors: Siva Reddy, New Delhi (IN); Leelawathi Sadhu, New Delhi (IN); Vijaya Shukla, Koln (DE); Giovanni Ferraiolo, Genis-Pouilly (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/149,533

(22) PCT Filed: Dec. 8, 2000
(86) PCT No.: PCT/EP00/12446

(30) Foreign Application Priority Data
Dec. 8, 1999 (GB) 9929075.1
Jul. 14, 2000 (GB) 0017369.0

(51) Int. Cl. C12Q 1/68

(52) U.S. Cl. 435/6

(57) ABSTRACT

The present invention relates to a method of obtaining a stable transplastome, which method comprises transforming a recipient plastome with a polynucleotide comprising: (a) a 5′ sequence homologous to a region of the recipient plastome, and, joined thereto; (b) a sequence heterologous to the recipient plastome comprising a coding region operably linked to at least one regulatory region capable of securing expression of the coding region in the plastid, and, joined thereto; (c) a 3′ sequence homologous to a region of the recipient plastome.

### FIELD OF THE INVENTION

The present invention is in the field of plant biotechnology. It relates more particularly to the stable transformation of a plastidic genome with a foreign polynucleotide, and to the generation of stable transplastomic plant cells, plants, seeds and plants of second and further generations.

### BACKGROUND OF THE INVENTION

### Plastids

Plastids are organelles found in plant cells and the cells of photosynthetic algae such as Chlamydamonas. Various kinds of plastids exist and are derived from undifferentiated plastids, termed proplastids. Differentiated plastids include amyloplasts, chromoplasts, chloroplasts, etioplasts and leucoplasts. Chloroplasts are the most common plastids, and are the site of photosynthesis. Each photosynthetic cell contains multiple chloroplasts, typically from 50 to 100. Chloroplasts have their own genome, the plastome, which exists in addition to the main cellular (nuclear) genome, and transcription and translation systems. The latter resemble prokaryotic transcription and translation systems. Each chloroplast contains multiple genome copies, typically from 50 to 100. A plastid genome, referred to as a plastome, comprises a double stranded circular DNA molecule.

### Drawbacks of Nuclear Transformation

In the field of biotechnology, the ability to express a foreign gene, referred to as transgene expression, in the organism of choice, is desirable. Typically, transgene expression in plants is achieved by the integration of a transgene construct into nuclear DNA. Due to the low copy number of native genes within the nuclear genome, the number of copies of a transgene in a nuclear transformed plant is typically low. Consequently the expression levels achieved by nuclear transformation is typically low. Expression of the transgene may also be affected by other factors, such as the site of transgene integration. This means that the levels of expression achieved by independently derived nuclear transformed plants harbouring the same transgene can be highly variable.

Plant zygotes contain nuclear DNA derived from both the female (ova) and male (pollen) gametes, both of which contribute to the characteristics of the mature plant. Therefore, nuclear-encoded transgene can be spread in the ecosystem by the dispersal of pollen, which contains the male gametes, from plants containing a nuclear transgene and subsequent fertilisation of wild type plants. The dispersal of pollen derived from a nuclear transformed plant, therefore, provides a potential vehicle for the unwanted (“lateral”) transmission of transgenes into other species. There is currently considerable concern about this, especially over the possible transmission of herbicide/insecticide/disease resistance traits from transgenic crops (typically cereals) to weedy relatives growing around the crop fields, leading to the possibility of resistant weeds (so-called “super-weeds”) which are hard to eliminate because of their resistant traits (Daniell, 1999).

### Advantages of Chloroplast Transformation

Many of the disadvantages of nuclear transformation can be avoided by targeting transgene integration to the plastome. A transformed plastome is referred to as a transplastome. Due to the existence of multiple plastome copies within each chloroplast, the copy number of an integrated transgene is high. This leads to a level of expression of a transplastomic gene that is typically higher than for an equivalent transgene integrated into nuclear DNA. Such plants are referred to as transplastomic plants. Plastids are maternally inherited. That is, zygotes derive plastids from the cytoplasm inherited from the female gamete, whereas pollen does not contribute plastids to the zygote. Pollen derived from transplastomic plants does not, therefore, contain the transgene and so transgene transmission to other species is not possible.

This is particularly beneficial in view of public fears related to the spread of transgenes and their potential impact on the ecosystem.

Foreign DNA has previously been introduced into chloroplasts using a biolistic method (Boynton et al, 1988; Svab et al,1990; Svab and Maliga 1993; U.S. Pat. No. 5,451,513; U.S. Pat. No.5,545,817; U.S. Pat. No.5,545,818; U.S. Pat. No.5,576,198; U.S. Pat. No.5,866,421) and a PEG-based procedure (Golds et al, 1993). Typically, the transgene in a chloroplast transformation vector is flanked by DNA regions homologous to regions of the plastome. These flanking regions enable the site-specific integration of the transgene construct into plastome by the process of homologous recombination, a process which naturally occurs in plastids. Therefore, the site of transgene integration is more assured in chloroplast-based techniques relying on homologous recombination than in nuclear-based processes. Therefore, more uniform transgene expression results between independently derived transplastomic plants than between independently derived nuclear transformed ones.

### Drawbacks of Chloroplast Transformation

It is clearly desirable to maximise the efficiency of transgene expression. For a transgene construct to be expressed, it is necessary for the coding region to be operably linked to regulatory regions, including a promoter, and typically, a terminator region. Chloroplast transcription and translation systems display species-specificity with respect to promoter recognition (Sriraman, 1998a). Since regulatory regions endogenous to the recipient plastome are known to function efficiently in that plastome, the regulatory regions used in transgene constructs are typically derived from sequences endogenous to the recipient plastome.

However, use of sequences endogenous to the recipient plastome in the transgene construct inevitably results in the existence of duplicated sequences within the transplastome, and therefore, internal homology exists. A recombination event between a transgene regulatory sequence and an endogenous plastomic sequence may then occur. Such an event can lead to genome rearrangements or large scale deletions in the transplastomic DNA (Svab and Maliga 1993). If such an event occurs at an early stage of transformation or at a later stage of plant development the transgene expression becomes unstable and may be lost entirely. Consequently, the techniques of plastome transformation known in the art do not provide for stable transplastomic gene expression.

Furthermore, although transplastomic plants typically express transgenes at higher levels than nuclear-transformed plants, recovery of commercially viable amounts of protein can be problematic. For a commercially viable biofarming, the recombinant protein, if needed, must be purified following cost-effective and rapid purification procedures. In plants, rapid purification is important because there are no protease(s) deletion host plants available in which to express recombinant proteins. Rapid purification of recombinant protein can be facilitated by using affinity-based chromatography if the expressed protein is engineered to contain a ligand at the N- or C-terminal ends (Chong et al, 1997; diGuan et al, 1988; Hochuli et al, 1987; Smith and Johnson 1988). Although this approach is commonly used in E. coli, it is rarely used in the purification of recombinant proteins expressed in transgenic plants (Flachmann and Kuhlbrandt 1996; Sugiura 1999).

Identification of recombinant protein-containing fractions by conventional methods such as SDS-polyacrylamide gel electrophoresis (SDS-PAGE), enzyme linked immunosorbant assay (ELISA) and Western blot analysis is time consuming and laborious. If the recombinant protein can be detected by simple and rapid tests, then purification may be achieved much faster. This is an important consideration for highly labile proteins.

### Drawbacks of Using Heterologous Sequences for Promoters

Attempts have been made to use “heterologous” regulatory regions, that is, regions derived from the plastome of a different species to the recipient plastome. However, in most cases, heterologous regulatory sequences were found to have no function. For example, a maize plastome promoter was shown to have no function in the plastome of Chlamydomonas (Blowers et al, 1989) which contrasts with the successful use of endogenous regulatory regions (Blowers et al, 1990). Notably for the present invention, Sriraman (1998b) demonstrated that a rice (a monocotyledonous plant) plastome promoter has no function in the plastome of Nicotiana tabacum (a dicotyledonous plant).

Two reports exist of the use of functional regulatory sequences derived from plastomes of a different species to the recipient plastome. WO 97/32977 teaches that the use of a Nicotiana tabacum plastome promoter in Arabidopsis thaliana and Brassica napus results in transplastomic expression. Sriraman et al, 1998 teaches that a plastomic Nicotiana tabacum promoter was able to drive expression of a transplastomic gene in Arabidopsis thaliana. Also a or Spinacia oleracea promoter was able to drive transplastomic expression in Nicotiana tabacum. However, the authors noted no particular advantage to this in terms of transplastomic stability. Stability of the transplastome was not tested. Furthermore, because inter-species homology exists between plastomes (Blowers et al, 1989), especially of related species, arbitrary selection of plastome regulatory regions from one species does not guarantee sufficient heterology to the plastome of another species. Therefore, the previous reports have provided no means of selecting appropriate regulatory regions for stable transplastomic expression.

This is further confirmed by other documents from the patent literature. For example, WO98/55595 provides certain plastid promoters for transgene expression in higher plant plastids. WO97/06250, originating from the same research group, relates to plastid transformation but without addressing the problems of endogenous sequence duplication by homologous recombination. The same applies to U.S. Pat. No. 5,932,479, which is directed towards the production of an expression cassette for reducing the number of cloning steps needed for transformation, but without addressing the problems of sequence duplication within the plastid and subsequent difficulities caused by homologous recombination.

In addition, WO98/11235 provides methods of expressiong celluloytic enzymes in plastids via inducible, transactivator-mediated system. Homologous recombination is said to result in stable integration of the transgene, though without recognition of the problems that duplication of endogenous plastome sequence in this manner can cause. Also, it is unclear and unpredictable whether the promoters used would actually have any substantial activity in plastids. WO97/32977, a previous application naming one the present Inventor as an Inventor, concerns plastid transformation of Arabidopsis, but not with a view to obtaining stable transformation or avoiding the problems of homologous recombination. Most transformants were actually nuclear-, not plastid-transformed, no fertile plants were obtained and no transmission of transgene to progeny demonstrated.

Finally, WO99/46394 provides a plastid transformation method in which a regulatory sequence having a reduced, but still high (up to 90%), identity to a native plastid regulatory sequence is used. However, the goal of WO99/46304 was to improve plastid transformation frequency, which was misdirected, as this is only an issue for nuclear transformation, where independently transformed plants exhibit different characteristics owing to site-specific recombination. According to the methods of the invention, which are independent of site-specific recombination, this is not a problem as independently transformed plants behave in the same manner. In WO99/46394, an Arabidopsis promoter was used in the transformation of a tabacco chloroplast, i.e. a dicot promoter in a dicot chloroplast Although WO99/46394 also notes the possibility of using an Arabidopsis (dicot) promoter in maize (a monocot), this was unsubstantiated, and of doubtful validity without experimental proof, in view of the previously noted failure of the maize rbcL promoter to function in the heterologous plant species Chlamydamonas (Blowers et al). In addition, WO99/46394 provides no confirmation beyond the seedling stage or of transmission of the transgene to progeny plants.

### SUMMARY OF THE INVENTION

Against this background the Inventors have, surprisingly, identified two promoter regions and two terminator regions from the plastome of rice which remain fully functional when integrated into the plastome of tobacco. Tobacco is a dicotyledonous plant, and is therefore, evolutionarily distant from rice, which is monocotyledonous. Previous reports (e.g. Blowers et al, 1989; Blowers et al, 1990; Sriraman et al, 1998b) teach that such divergent sequences would be unlikely to function and so this result is surprising. In fact, the Inventors have shown that the rice promoters integrated into the tobacco plastome function in an identical manner to when in the rice plastome. Further tests performed by the Inventors have demonstrated that tobacco plastomes transformed with heterologous transgene constructs containing these regulatory regions do not undergo additional internal recombination and remain stable, even in later generations of the transplastomic plants. Expression analysis has demonstrated that these transplastomic plants are able to express the transgene to levels of from 100-fold to 5000-fold higher than nuclear transformed plants.

The present invention provides a method of providing high, uniform, reliable expression of transgenes in plants, with stable inheritance of the trait whilst avoiding the potential for the dangerous spread of transgenes to the ecosystem. This is achieved by generating transplastomic plastids, plant cells and plants of first and subsequent generations with assured transplastome stability. By developing a greater understanding of the requirements for a stable transplastome and of plastome regulatory regions, the Inventors have developed a strategy for the selection of suitable transgene constructs with which to generate stable transplastomes.

In one embodiment of the invention, heterologous plastome-derived promoters are used to generate transforming polynucleotides. Promoters that contain well defined −10 and −35 sequence motifs, and show sequence heterology to the proposed recipient plastid, represent suitable candidates. The invention further provides a vector for the convenient insertion of regulatory and coding sequences of choice to allow the definitive testing of suitable candidates. The Inventors' unexpected findings allow the selection of truly heterologous regulatory regions able to function in the stable transplastomic expression of transgenes in a plant species of choice.

Another embodiment of the invention provides transforming polynucleotides comprising homologous regulatory elements, yet integration of the polynucleotide into the plastome result in a stable transplastome. This is achieved by positioning homologous regulatory sequences within the homologous flanking sequences. Thus the process of homologous recombination prevents duplication of endogenous plastomic sequence, and the transplastome is stable.

Another embodiment of the invention provides transforming polynucleotides wherein the coding region is not linked to a regulatory sequence, yet stability and expression of the transgene is secured in the transplastome. By designing homologous flanking regions so that homology exists with expressed regions of the plastome, then following integration, endogenous plastome regulatory regions can be used to secure expression of the coding region within the transforming polynucleotide.

Accordingly the invention provides:

A method of obtaining a stable transplastome, which method comprises transforming a recipient plastome with a polynucleotide comprising:

(a) a 5′ sequence homologous to a region of the recipient plastome, and, joined thereto;

(b) a sequence heterologous to the recipient plastome comprising a coding region operably linked to at least one regulatory region capable of securing expression of the coding region in the plastid, and, joined thereto; and

(c) a 3′ sequence homologous to a region of the recipient plastome.

The invention also provides:

A method of obtaining a stable transplastome, which method comprises transforming a recipient plastome with a polynucleotide comprising:

(a) a 5′ sequence homologous to a region of the recipient plastome, and, joined thereto;

(b) a sequence heterologous to the recipient plastome comprising a coding region, and, joined thereto;

(c) a 3′ sequence homologous to a region of the recipient plastome; wherein the coding region defined in (b) is operably linked to at least one regulatory region capable of securing expression of the coding region in the plastid, which regulatory region is homologous to a region of the recipient plastid and is positioned in homologous region (a) or (c).

The invention also provides:

A method of obtaining a stable transplastome, which method comprises transforming a recipient plastome of a multicellular organism with a polynucleotide comprising:

(a) a 5′ sequence homologous to a region of the recipient plastome, and, joined thereto;

(b) a sequence heterologous to the recipient plastome comprising a coding region, and, joined thereto;

(c) a 3′ sequence homologous to a region of the recipient plastome; wherein, following transformation, the coding region defined in (b) is operably linked to at least one regulatory region capable of securing expression of the coding region in the plastid, which regulatory region is an endogenous plastome regulatory region positioned 5′ to homologous region (a) or 3′ to homologous region (c) within the transplatome.

The invention also provides:

A method of obtaining a transplastomic plastid, which method comprises transforming a plastome within a plastid by a method according to the invention.

The invention also provides:

A method of obtaining a transplastomic cell, which method comprises transforming a plastome within a plastid within a cell by a method according to the invention. The invention also provides:

A method of obtaining a homotransplatomic cell, which method comprises obtaining transplastomic cells by a method according to the invention and selecting for the presence of the transplastome.

The invention also provides:

A method of obtaining a first-generation transplastomic or homotransplastomic plant, wherein the method comprises regenerating a transplastomic or homotransplastomic plant cell obtainable by the method of the invention to give a transplastomic or homotransplastomic plant.

The invention also provides:

A method of obtaining a transplastomic or homotransplastomic plant seed, wherein the method comprises obtaining a transplastomic or homotransplastomic seed from a transplastomic or homotransplastomic plant obtainable by a method of the invention.

The invention also provides:

Use of a polynucleotide as defined herein, in the production of a stable transplastome, a transplastomic or homotransplastomic plastid, a transplastomic or homotransplatomic cell, a transplastomic or homotransplastomic plant or a transplastomic or homotransplastomic seed.

The invention also provides:

A transplastome transformed with a polynucleotide as defined herein.

The invention also provides:

A transplastomic or homotransplastomic plastid comprising a transplastome according to the invention.

The invention also provides:

A transplastomic or homotransplastomic plant cell, callus, first-generation plant, obtainable from a cell according to the invention or a transplastomic or homotransplastomic seed, second-generation progeny plant or a plant of one or more further generations obtainable therefrom.

The invention also provides:

A method of obtaining a crop product comprising harvesting a crop product from a cell or plant obtainable by a method according to the invention and optionally further processing the harvested product.

The invention also provides:

A crop product obtainable by a method as just described.

The invention also provides:

Use of a vector for the generation of a stable transplastome, which vector comprises:

(a) a 5′ sequence homologous to a region of the recipient plastome comprising a unique restriction site at its 5′ terminus, and joined at the 3′ terminus by a unique restriction site to;

(b) a sequence heterologous to the recipient plastome comprising:

(i) coding regions for at least one selectable or scorable marker operably linked to regulatory regions capable of securing expression of the coding sequence in the transplastome; and optionally

(ii) regulatory regions capable of securing expression of a coding sequence in the transplastome, wherein each regulatory region comprises unique restriction sites at 5′ and 3′ borders; and optionally

(iii) a coding sequence operably linked to the regulatory regions of wherein the 3′ terminus of the heterologous sequence is joined by a unique restriction site to;

(c) 3′ sequence homologous to a region of the recipient plastome comprising a unique restriction site at its 3′ terminus.

### BRIEF DESCRIPTION OF THE DRAWINGS

FIG. 1A: Site-specific integration of uidA and aadA genes into plastid genome. Arrows with in the boxes indicate the direction of transcription. Crossed lines indicate the two homologous recombinations necessary to integrate the transgenes.

FIG. 1B: Site-specific integration of transgenes into plastid genome through homologous recombinations. Arrows indicate the direction of transcription. Crossed lined indicate the site of recombinations. (B) Possible mechanism of rearrangements/deletions in the transgenic tobacco genome due to duplication of 5′ and 3′ regulatory regions when derived from homologous source involving homologous recombinations. The Nt. 326TS are derived when the vector, pVSR326TS containing a selectable uidA marker gene (for simplification the selectable aadA gene was not shown) under the regulation of 5′ and 3′ psbA regions taken from tobacco plastid genome were introduced into wild-type tobacco chloroplasts. The integration is facilitated through two homologous recombinations involving rbcL gene sequences on one side and accD gene sequences on the other side. About 10-50% of the transformed plants contain altered genome due to further homologous recombinations between the introduced and endogenous 5′ and 3′ regulatory sequences depending on the number of recombinations. As result, the native psbA or introduced uidA or a large portion of plastid genome may be deleted, which can be seen phenotypically as white chlorophyll deficient patches in the leaf making the plants photosynthetically incompetent

FIG. 2: Construction of novel plastid transformation vectors utilizing heterologous 5′ and 3′ regulatory sequences from rice (a monocot) to transform tobacco (a dicot). Detailed stepwise construction of vector, pVSR326.

FIG. 3: Vector maps and the site-specific integration of aadA and uidA genes. (A) The pVSR326 transformation vector and recombination with wild type plastid genome (cp DNA) in transplastomic plants (Nt 326-37) through two homologous recombination events at targeting sequence (shaded boxes). The chimeric uidA gene expression is under the regulation of rice psbA gene promoter (psbARP) and it's 3′ UTR (psbART). The chimeric aadA gene expression is under the regulation of modified rice rrn operon promoter (16SRP) and the 3′ UTR of rice rbcL gene (rbcLRT). The expected size of the DNA fragments from various restriction enzyme digestions are shown in solid arrows. In addition, the direction and expected size of the transcripts are shown in dotted arrows. Relevant restriction sites are mentioned. (B) The pVSR326S transformation vector and recombination with wild type plastid genome (cp DNA) in transplastomic plants (Nt 326S-1) through two homologous recombination events at targeting sequence (shaded boxes). The chimeric uidA gene expression is under the regulation of rice psbA gene promoter (psbARPS) and its 3′ UTR (psbART). The chimeric aadA gene expression is under the regulation of modified rice rrn operon promoter (16SRP) and the 3′ UTR of rice rbcL gene (rbcLRT). (C) The pVSR326T transformation vector and recombination with wild type plastid genome (cp DNA) in transplastomic plants (Nt 326T-1) through two homologous recombination events at targeting sequence (shaded boxes). The chimeric uidA gene expression is under the regulation of tobacco psbA gene promoter (psbATP) and its 3′ UTR (psbATT). The chimeric aadA gene expression is under the regulation of modified tobacco rrn operon promoter (16STP) and the 3′ UTR of tobacco rbcL gene (rbcLT). (D) The pVSR326TS transformation vector and recombination with wild type plastid genome (cp DNA) in transplastomic plants (Nt 326TS-1) through two homologous recombination events at targeting sequence (shaded boxes). The chimeric uidA gene expression is under the regulation of tobacco psbA gene promoter (psbARPS) and its 3′ UTR (psbART). The chimeric aadA gene expression is under the regulation of modified tobacco rrn operon promoter (16STP) and the 3′ UTR of tobacco rbcL gene (rbcLTT).

FIG. 4: General scheme that was followed to generate the chloroplast transformed plants using gene gun method.

FIG. 5: Southern hybridization analysis to show the site-specific integration of chimeric aadA and uidA genes into plastid genome. About 3 μg of total genomic DNA from each sample was digested with NcoI-SacI and probed with uidA coding region. DNA digested with BamHI-XhoI and probed with aadA coding region. DNA digested with ClaI and probed with targeting sequence (rbcL-accD). The uidA and aadA probes revealed the presence of chimeric genes in plastid genome. The targeting sequence probe revealed the sitespecific integration and the homoplasmy for the introduced genes. Genomic DNA from wild type plant was also included as control.

FIG. 6: Northern blot analysis showing the steady state levels of aadA and uidA transcripts. About 3 μg of total RNA was electrophoresed, blotted and hybridized to nick translated aadA/uidA/psbA/16S rDNA probes. RNA from wild type tobacco; Nt. 326-37 and Nt. 326T-1 were analysed. The same blot was reprobed after stripping the probe each time.

FIG. 7: Mapping of the 5′ ends of the chimeric uidA and aadA transcripts by primer extension. (A) Lanes 1-4 show partial nucleotide sequence of rice chloroplast primary clone, pRP7, generated using SR16 primer. Lanes 7-10 show partial nucleotide sequence of pVSR326 plasmid generated using SR14 primer. Lane 5 and 6 show the extension product of primer SR16 using total RNA from wild type rice and tobacco leaves, respectively. Lanes 11 and 12 show the extension product of primer SR14 using total RNA from transplastomic line (Nt 326-37) and wild type tobacco, respectively. (B) Lanes 14 and 8-11 show partial nucleotide sequence of plasmid pVSR326 generated using SR16 and SR14 primers, respectively. Lanes 5-7 show the extension products of primer SR02 using total RNA from transplastomic line (Nt 326-37), wild type tobacco and rice plants, respectively. Lanes 12 and 13 show the extension products of primer SR15 using total RNA from transplastomic line (Nt 326-37) and wild type tobacco, respectively. The nucleotide sequence surrounding the transcription initiation is shown and the −10 sequence motif is boxed. The numbers 118 and 77 in brackets indicates the position of nucleotide from mature 16S rRNA and translation initiation ATG codon of Prm and psbA genes of rice, respectively. The ATGC represent the sequencing reaction and R, T and P represent RNA from rice, tobacco and transplastomic line (Nt 326-37) used in the primer extension reaction.

FIG. 8: Western blot analysis for detection of GUS protein. Ten micrograms of leaf soluble protein was analyzed by Western blot analysis. Protein extract from transformed transgenic plants, Nt. 326-37, Nt. 326T-1, Nt. 121-1 and wild type. Note the presence of expected (68 kDa) GUS protein band in Nt. 326-37 and Nt. 326T-1 plants.

FIG. 9: Comparison of GUS (A) and aadA activity (13) in the transformed plants using different vectors. (A) GUS activity was compared among a nuclear transformed plant (Nt. 121-1) with pBI 121 vector (Clonetech), chloroplast transformed plants with vector pVSR326 (Nt. 326-37) and with the vector pVSR326T (Nt. 326T-1). Note that the chloroplast transformed plants have several fold high expression when compared to nuclear transformed plant. Also note that there is no significant difference in the expression levels of GUS between Nt. 326-37 (uidA is under the regulation of rice 5′ and 3′ psbA region) and Nt.326T-1 (uidA is under the regulation of tobacco 5′ and 3′ psbA region).

The aadA activity was compared between the Nt. 326-37 (aadA is under the regulation of rice 5′ rrn region) and Nt. 326T-1 (aadA is under the regulation of tobacco 5′ rrn region). There is no significant difference in the expression levels of aadA between Nt. 326-37 and Nt.326T-1.

FIG. 10: Southern hybridization analysis to show the inheritance of stable plastid DNA among the progeny. The total DNA sample was digested with EcoRI (A) or BamHI (B) and hybridized with psbA and 16S rDNA (including regulatory sequences), respectively. Arrow indicate the expected size signal in the absence of any rearrangements in the genome. Note the uniform hybridization pattern in all the transformed lines indicating the stable plastid genome among the progeny. Lane 1 represent the DNA sample from wild type plant and lanes 2-29 represent progeny from twenty-eight independently transformed plants.

FIG. 11: Maternal inheritance of aadA and the detection of altered phenotypes among the progeny derived from Nt. 326-37 and Nt. 326TS-1 plants. Seeds were germinated on spectinomycin (500 mg/L) containing RM plates. The progeny that received aadA will remain green where as the progeny that did not receive aadA due to lack of transmission through pollen or deletions in the genome turn to white. Note the presence of only white seedlings in the progeny derived from a cross where transformed plant was male and the wild type plant was female. Also note the presence of white seedlings among the progeny derived from self fertilized or between a cross involving transformed plant as female and wild type as male indicating the unstable genome when transformed with vectors containing regulatory regions from homologous source.

FIG. 12A: Representative plants having altered phenotype due to rearrangements/deletions. Plants with such phenotye was commonly observed when pVSR326T or pVSR326TS vectors were used to transform the plants. Note the chlorophyll deficient areas in the leaf. Chlorophyll deficient patches are phenotypic indication of altered genome due to undesirable recombinations between introduced and native regulatory regions. Note the development of albino side branches from a green transgenic plant (C, transformed with pVSR326T and D, F transformed with pVSR326TS) indicating that homologous recombinations are unpredictable in these transgenic plants.

FIG. 12B: Representative leaves from the transformed plants having altered phenotype due to rearrangements/deletions. Plants with such phenotye was commonly observed when pVSR326T or pVSR326TS vectors were used to transform the plants. Note the chlorophyll deficient areas in the leaf.

FIG. 13: Transformation and expression of ifnG in the tobacco nuclear genome. A). Map of the vectors pBI121 and pBIIFNG. Double head arrows indicate the size of DNA fragments after the restriction digestion with PstI (P), XbaI (X) and EcoRI (E). LB and RB represent left and right border sequences of transformed DNA (T-DNA) of Agrobacterium. Dashed arrow indicates the direction and size of the transcript. 35SP: CaMV 35S promoter, NPTII: neomycin phosphotransferase II that confers resistance to kanamycin, uidA: β-glucuronidase (GUS) reporter gene. B). Southern hybridization of genomic DNA isolated from wild type (1), Nt. 121-1 (2), Nt. BITFNG-1 (3), Nt. BIITFNG-2 (4) plants was digested with PstI+EcoRI (P+E), XbaI (X) and probed with ifnG and uidA radiolabeled probes. C). RT-PCR analysis of RNA from wild type (1 &3) and Nt. BIIFNG-1 (2 & 4) plants using ifnG-uidA gene specific primers (1 & 2) and ifnG gene specific primers (3 & 4).

FIG. 14: Restriction map of transformation vectors used to express IFN-g in tobacco chloroplasts and the ifnG gene. A). Vector pVSR326 contained aadA selectable marker that confers resistance to spectinomycin under the control of rice rrn promoter (rrnP) and uidA reporter gene under the regulation of rice psbA promoter (psbAP). Double head arrow indicate the size of the DNA fragment expected when digested with ClaI (C). The chimeric uidA and aadA genes were flanked by tobacco rbcL and accD gene sequences for site-specific integration into tobacco plastid genome. Dashed arrow indicates the direction and size of the uidA transcript. B). Restriction map of vector p326IFNG, partial chloroplast DNA of tobacco (cpDNA) and the transformed tobacco plant (Nt. 326IFNG-1) plastid DNA. Double head arrows indicate the size of DNA fragments after the restriction digestion with ApaI (A) and ClaI (C) alone and together. Position of the His-tag (6×(His) and factor Xa site (Fa-Xa) are indicated. Dashed arrow indicates the direction and size of the ifnG transcript. A possible mechanism for site-specific integration of aadA and ifnG through two homologous recombinations (crossed lines) were also shown C). Nucleic acid and deduced amino acid sequence of ifnG used to express recombinant IFN-g in tobacco chloroplasts. Sequences coding for His-tag, factor Xa and various restriction enzymes are also indicated. D). Restriction map of vector pGUSIFNG, partial chloroplast DNA of tobacco (cpDNA) and the transformed tobacco plant (Nt. GUSIFNG-1) plastid DNA. Double head arrows indicate the size of DNA fragments after the restriction digestion with XhoI (Xh), ClaI (C), BamHI (B), XbaI (X) alone or in combination. Position of the His-tag (6×(His) and factor Xa site (Fa-Xa) are indicated. Dashed arrows indicate the direction and size of the uidA:ifnG and aadA transcripts. A possible mechanism for site-specific integration of aadA and uidA:ifnG through two homologous recombinations (crossed lines) were also shown.

FIG. 15: Southern and Northern blot analysis to confirm the stable integration and expression of aadA, ifnG, uidA and uidA:ifnG genes in tobacco chloroplasts. A). Genomic DNA isolated from wild type (1) and six independently transformed (2-7) plants was digested with ClaI and hybridized with rbcl-accD targeting DNA probe. B). Genomic DNA isolated from Nt. 326IFNG-1 (1), Nt. 326IFNG-2 (2) and wild type (3) plants was digested with ClaI (C), ApaI+XbaI (A+X), ApaI (A) enzymes and probed with aadA and ifnG gene probes. C). Genomic DNA isolated from Nt. GUSIFNG (1) and wild type (2) plants was digested with ClaI (C), XhoI (Xh), BamHI (13), NcoI XbaI (N+X) and probed with aadA, ifnG, uidA and rbcL-accD gene sequences. D). Total RNA isolated from wild type (1), Nt. 326IFNG-1 (2), Nt. 326IFNG-2 (3) plants were separated on agarose gel, blotted on to nylon membrane and hybridized with ifnG probe. Inset at the bottom shows the hybridization of the same blot with 16S rDNA probe as a loading control. E & F). Blots containing total RNA from Nt. GUSIFNG-1(1), Nt. GUSIFNG-2 (2), Nt. 326-37 (3) and wild type (4) plants were hybridized with uidA (E) and ifnG (F) probes.

FIG. 16: Analysis and purification of recombinant IFN-g expressed in tobacco chloroplasts. A). Western blot analysis of total soluble proteins from wild type (1) and Nt. 326IFNG-1 (2) plants were probed with anti-His and anti-IFN-g antibodies. Proteins were partially purified on Ni-NTA column and 10 μg of protein was loaded in each lane. Arrow indicates the presence of an expected size protein. B). Western blot analysis of total soluble proteins from Nt. GUSIFNG-1 (1), Nt. 326-27 (2) and wild type (3) plants were probed with anti-GUS antibodies. About 10 μg protein from crude extracts was loaded in each lane. Arrows indicate the presence of expected size GUS and GUS:IFN-g fusion proteins. C).Estimation of half-life for GUS and IFN-g proteins expressed in tobacco chloroplasts. From a defined intervals, pulse labeled proteins were immunoprecipitated with anti-GUS or anti-IFN-g, separated on SDS-PAGE and exposed to autoradiography. The intensity of the signal was quantified and data plotted as graph. Arrows indicate half-life of both the proteins. D). The SDS-PAGE analysis of Ni-NTA fractions showing highly purified single band of 85 kDa protein corresponding to the size of GUS:IFN-g fusion protein, as judged by commassie blue stain. Activity of the GUS is correlated with the amount of the protein present in the fractions. E). Western blot showing the cross reactivity of Ni-NTA purified GUS:IFN-g fusion protein (1) and recombinant IFN-g separated from the GUS fusion partner and purified further using—Sepharose column (2) with anti-IFN-g antibodies. F). Antiviral activity of the purified recombinant IFN-g. The human lung carcinoma cells precultured for 24 h in the presence (top) or absence (bottom) of rh-IFN-g were challenged with 104 PFU of encephalomyocarditis (EMC) virus. A 24 h pretreatment of rh-IFN-g offered complete protection whereas the untreated cell were infected and disintegrated.

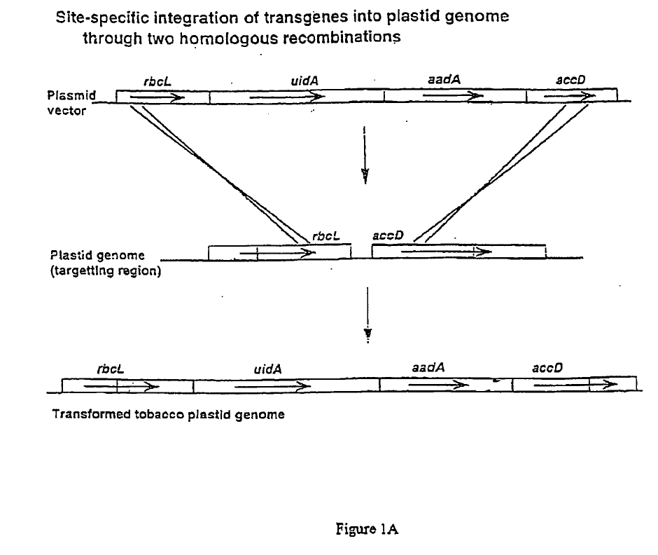

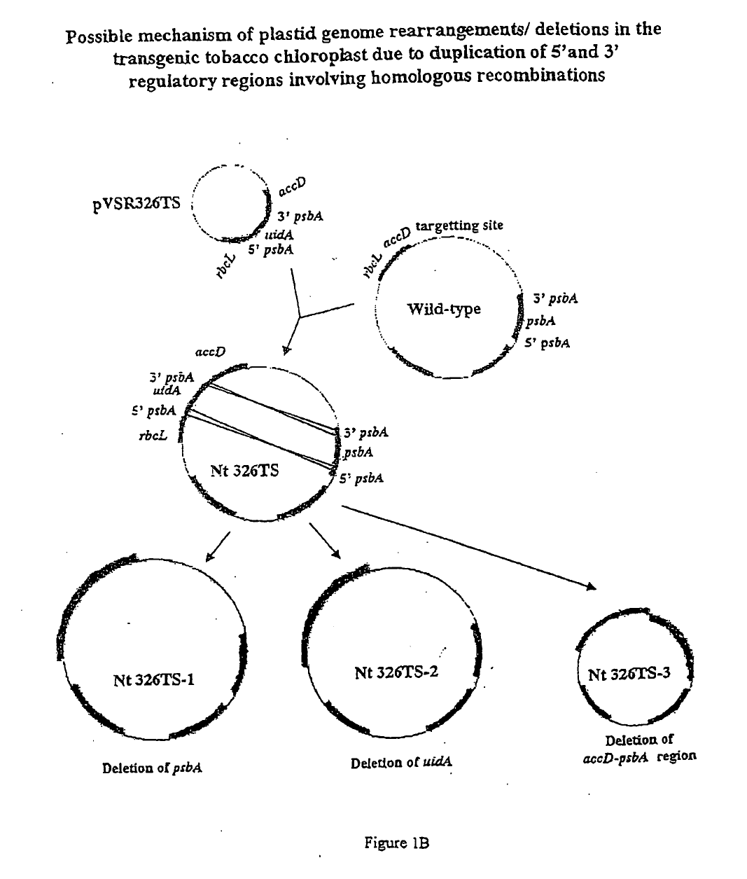

### DETAILED DESCRIPTION OF THE INVENTION

### Plastids

Plastids suitable for use in this invention may be derived from any organism that has plastids, preferably a multicellular organism that has plastids. They may be derived from any cell type and may be of any differentiated or undifferentiated state. Such states include undifferentiated proplastid, amyloplast, chromoplast, chloroplast, etioplast, leucoplast. Preferably, the plastid will be a chloroplast.

Plastids comprise their own genome, herein referred to as a plastome. Typically individual plastids comprise multiple plastomes, more typically from 5 to 500, most typically from 50 to 100. Herein, a recipient plastome is one that may be transformed with a transforming polynucleotide of the invention, as described below. Recipient plastomes for use in the invention may be isolated from natural sources, or may be artificially generated by techniques known in the art, such as recombinant techniques, random mutagenesis, site directed mutagenesis, or other alterations. Alterations may include additions, insertions, deletions, substitutions or inversions. Herein, a recipient plastome transformed with a transforming polynucleotide according to the invention is referred to as a transplastome. Plastids comprising a transplastome are referred to as transplastomic. Plastids wherein all plastomes are identical, or substantially identical, transplastomes are referred to as homotransplastomic. In this context, the plastomes of plastids are substantially identical if they all comprise the coding region of the transforming polynucleotide of the invention, and preferably any associated regulatory sequences, or at least enough of the coding regulatory sequences to secure expression of the coding sequence. Cells containing plastids are homotransplastomic if all the plastids in the cell are homotransplastomic. Plants, plant parts and seeds are homotransplastomic if all of their cells are homotransplastomic.

### Recipient Plastomes

Suitable sources of recipient plastome include plants, for example, spernatophytes, pteridophytes (ferns, clubmosses, horsetails), bryophytes (liverworts and mosses), and algae. Typically the recipient plastome will be a plastome of a multicellular organism, usually a spermatophyte. The plastome may be a plastome of any gymnosperm or an angiosperm. Suitable gymnosperms include conifers (such as pines, larches, firs, spruces and cedars), cycads, yews and ginkgos. More typically the recipient plastome is an angiosperm plastome and is of a monocotyledonous or dicotyledonous plant, preferably a crop plant Preferred dicotyledonous crop plants include tomato; potato; sugarbeet cassava; cruciferous crops, including oilseed rape; linseed; tobacco; sunflower; fibre crops such as cotton; and leguminous crops such as peas, beans, especially soybean, and alfalfa. Tobacco is particularly preferred. Preferred monocotyledonous plants include graminaceous plants such as wheat, maize, rice, oats, barley, rye, sorghum, triticale and sugar cane. Rice is particularly preferred. Alternatively, plastomes and regions of transforming construct for use in the methods of the invention may be recombinant or entirely synthetic in origin.

### Further Scope of the Invention

In a further embodiment of the invention, the transforming polynucleotide is adapted for use in the stable transformation of other organelles comprising genomes, such as, mitochondria. The skilled person will readily appreciate that the methods described above are equally applicable to the production of transgenic mitochondrial genomes, transgenic mitochondria, cells, calli, plants and seeds comprising stable transgenic mitochondria. Furthermore, the production of stable transgenic mitochondria is possible in all eukaryotic organisms.

### Generating a Stable Transplastome

The present invention provides a method for the production of stable transplastomes. The term stable, as used herein, refers to a transplastome in which internal recombination is not detectable over a period of time. Preferably, stability will be manifest by a lack of internal recombination within the transplastome after at least one cell division, for example, after up to ten cell divisions, or after up to one hundred cell divisions or more either in culture or during and/or after regeneration to give a first-generation plant. More preferably the stability is also retained in the second-generation plants that are progeny of the first-generation one and further progeny such as third-, fourth-, fifth- or sixth-generation plants. Suitable techniques for the detection of recombination in a transplastome are known in the art, for example, Southern analysis utilising polynucleotide probes appropriate for hybridisation to the transforming polynucleotide.

### Transforming Polynucleotides with Heterologous Regulatory Regions

The methods of the invention are based on a development of greater understanding of the requirements for a stable transplastome and plastome regulatory regions and provides polynucleotides suitable for carrying out the invention.

In one embodiment of the invention, a method is provided for obtaining a stable transplastome. The method comprises transforming a recipient plastome with a transforming polynucleotide comprising:

(a) a 5′ sequence homologous to a region of the recipient plastome, and, joined thereto;

(b) a sequence heterologous to the recipient plastome comprising a coding region; and joined thereto;

(c) a 3′ sequence homologous to a region of the recipient plastome.

### Homologous Regions

The transforming polynucleotide comprises homologous regions (a) and (c), which exist as flanking regions of the polynucleotide, that is, they define the 5′ and 3′ ends of the transforming polynucleotide. The homologous flanking regions allow insertion of the polynucleotide into the recipient plastome by homologous recombination.

Methods of measuring nucleic acid homology are well known in the art. For example the UWGCG Package provides the BESTFIT program which can be used to calculate homology (Devereux et al, 1984) e.g. in the context of whether sequences are homologous or heterologous for the purposes of the invention.

Similarly, the PILEUP and BLAST algorithms can be used to line up sequences (for example as described in Altschul, 1993; Altschul et al, 1990). Many different settings are possible for such programs. According to the invention, the default settings may be used.

In more detail, the BLAST algorithm is suitable for determining sequence similarity and it is described in Altschul et al, 1990. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 1 1, the BLOSUM62 scoring matrix (see Henikoff and Henikoff 1992) alignments (13) of 50, expectation (E) of 10, M=5, N=4, and a comparison of both strands.

The BLAST algorithm performs a statistical analysis of the similarity between two sequences; see e.g., Karlin and Altschul (1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a fused gene or cDNA if the smallest sum probability in comparison of the test nucleic acid to a fused nucleic acid is less than about 1, preferably less than about 0.1, more preferably less than about 0.01, and most preferably less than about 0.001.

The sequence of the homologous flanking regions comprise sequences homologous; or substantially homologous, to regions of the recipient plastome. Typically the homologous flanking regions comprise sequences at least 80% homologous to regions of the recipient plastome. Preferably the degree of homology will be at least 90%, most preferably 100%. The homologous flanking regions (a) and (c) may be homologous to the same, overlapping, coterminous, or distinct regions of the recipient plastome. The homologous flanking regions may be homologous to any regions of the recipient plastome, preferably to regions comprising a gene, pseudogene or intergenic sequence. When a homologous flanking region is homologous to a region of the recipient plastome comprising a gene, it is preferably homologous to regions comprising regulatory regions, coding regions, or intronic regions.

### Heterologous Regions

The transforming polynucleotide further comprises a heterologous region (b) between the 5′ and 3′ homologous flanking regions (a) and (c). The heterologous region (b) does not posses substantial homology to any region of the plastome and, when integrated, therefore remains stable within the transplastome. In a preferred embodiment, the heterologous region has at most 80% homology with any region of the recipient plastome. More preferably the heterologous region has at most 70%, 60% or 50% with any region of the recipient plastome.

### Coding Sequences

Typically the heterologous region (b) comprises at least one, preferably two, three or more, coding sequences, that is, sequences capable of being transcribed by the transcriptional mechanisms endogenous to the recipient plastid. A coding sequence may encode, for example, an mRNA, a tRNA, an rRNA, a ribozyme, or any other form of RNA known in the art. Preferably the coding sequence encodes an mRNA More preferably, the mRNA is capable of being translated by the translation mechanisms endogenous to the recipient plastid. Typically the mRNA is monocistronic or polycistronic.

The encoded polypeptide may have any function. The naturally occurring form of the protein encoded by the coding sequence is typically an extracellular protein (e.g. a secreted protein), an intracellular protein (e.g. cytosolic, organellar, plastidic, nuclear or membrane protein) or a protein present in the cell membrane. The naturally occurring form may be constitutively expressed or be tissue specific.

Functions of polypeptides encoded by the coding region may include herbicide, insecticide or disease resistance. Preferred herbicide resistance genes may be responsible for, for example, tolerance to: Glyphosate (e.g. using an EPSP synthase gene (e.g. EP-A-0 293,358) or a glyphosate oxidoreductase (WO 92/000377) gene); or tolerance to fosametin; a dihalobenzonitrile; glufosinate, e.g. using a phosphinothrycin acetyl transferase (PAT) or glutamine synthase gene (cf. EP-A-0 242,236); asulam, e.g. using a dihydropteroate synthase gene (EP-A-0 369,367); or a sulphonylurea, e.g. using an ALS gene); diphenyl ethers such as acifluorfen or oxyfluorfen, e.g. using a protoporphyrogen oxidase gene); an oxadiazole such as oxadiazon; a cyclic imide such as chlorophthalim; a phenyl pyrazole such as TNP, or a phenopylate or carbamate analogue thereof; spectinomycin e.g using the aadA gene, as exemplified below.

Insect resistance may be introduced, for example using genes encoding Bacillus thuringiensis (Bt) toxins. Likewise, genes for disease resistance may be introduced, e.g. as in WO91/02701 or WO95/06128.

The coding region of the polynucleotide of the invention may comprise a selectable marker gene i.e. marker genes that allow transformed cells to survive in the presence of agents that kill non-transformed cells. Any selectable marker gene may be used in the transforming polynucleotide of the invention. Typically, herbicide resistance genes, e.g. as defined above, may be used as selectable markers. Alternatively, coding regions that encode products which provide resistance to aminoglycoside antibiotics may be used as selectable marker, for example, encoded products that provide resistance to kanomycin, neomycin or chloramphenicol. The encoded polypeptide may cause morphological alterations to cultured transformed cells, such as isopentyltransferase (Kunkel et al, 1999). The encoded polypeptide may be a scorable marker, which allows transformed cells to be distinguished from non-transformed cells, generally by alteration of the transformed cell's optical properties. Any scorable marker may be used. Preferred scorable markers include, polypeptides which are able to alter the appearance or optical properties of transformed cells, for example: β-glucoronidase (i.e. the udiA:GUS gene); fluorescent proteins such as green fluorescent protein (GFP), yellow fluorescent protein (YFP) or cyan fluorescent protein (CFP); or luminescent proteins such as luciferase or aequorin. Cells with scorable optical differences can be sorted using techniques such as fluorescence activated cell sorting (FACS). In a preferred embodiment, the polynucleotide of the invention comprises a selectable marker and a scorable marker, for example, the FLARE-S marker genes which comprise aadA and GFP (Khan and Maliga, 1999).

The coding region of the polynucleotide of the invention may encode a protein with an role in a metabolic pathway, preferably a chloroplastic metabolic pathway, for example the photosynthetic metabolic pathway. Typically the expression of the encoded protein may alter flux in the pathway, for example the encoded protein may alter the photosynthetic ability of the transformed plant.

For example, chlorophyll biosynthesis in lower organisms is light-independent due to the presence of an enzyme that comprises three potypeptides encoded by the chlL, chlN and chlB genes. However chlorophyll biosynthesis in angiosperms is dependent on the presence of light and therefore darkness results in low chlorophyll biosynthesis. This problem could be overcome by the generation of a stable transplastomic angiosperm that expresses any or all of the chlL, chlN and chlB genes.

Furthermore, the ability of the photosynthetic enzyme RUBISCO to fix carbon dioxide varies amongst plants that belong to distinct taxanomic groups such as algae, bryophytes, gymnosperms and angiosperms. The level of carbon dioxide fixation could be modified by transformation of a recipient plastome from one organism with the chloroplastic gene or genes encoding the RUBISCO large subunits from another organism.

Additionally, many of the chloroplastic processes, including metabolic and signalling pathways, can be controlled by post-translational protein modification such as glycosylation or phosphorylation. These levels could be manipulated in the chloroplast by transforming a plastome with a gene or genes encoding enzymes responsible for glycosylation, deglycosylation, phosphorylation or dephosphorylation, thus allowing promotion of desirable pathways or inhibition of undesirable pathways.

Alternatively, expression of the encoded protein may introduce a new metabolic step or steps to the transformed organism. In one example of this, biodegradable plastics can be produced in the chloroplast by transforming the plastome with prokaryotic genes known in the art. As another example, the genes that control plastid division could be introduced into a plastome to alter the number of plastids within a cell, with concomitant modification in their associated processes, such as photosynthesis.

Alternatively the encoded polypeptide may be expressed to enable its mass production, and have no particular relation to the biological processes of the plastid, cell or organism in which it is expressed. It may be any polypeptide known in the art. It may be derived from any organism, preferably from a prokaryote, fungus, plant or animal. Typically the polypeptide may be derived from a human. The polypeptide may perform any function in vivo. The protein may be a blood protein, such as a clotting protein (e.g. kinogen, prothrombin, fibrinogen factor VII, factor VIII or factor IX). The protein may be an enzyme, such as a catabolic or anabolic enzyme. The enzyme may be a gastro-intestinal enzyme, metabolic (e.g. glycolysis or Krebs cycle) enzyme or a cell signalling enzyme. The enzyme may make, breakdown or modify lipids, fatty acids, glycogen, amino acids, proteins, nucleotides, polynucleotides (e.g. DNA or RNA) or carbohydrate (e.g. sugars), and thus may typically be a protease, lipase or carbohydrase. The enzyme may be a protein modifying enzyme, such as an enzyme that adds or takes chemical moieties from a protein (e.g. a kinase or phosphatase).

The protein may be a transport or binding protein (e.g. which binds and/or transports a vitamin, metal ion, amino acid or lipid, such as cholesterol ester transfer protein, phospholipid transfer protein or an HDL binding protein). The protein may be a connective tissue protein (e.g. a collage, elastin or fibronectin), or a muscle protein (e.g. actin, myosin, dystrophin or mini-dystrophin). The protein may be a neuronal, liver, cardiac or adipocyte protein. The protein may be cytotoxic. The protein may be a cytochrome.

The protein may be able to cause the replication, growth or differentiation of cells. The protein may be development gene (e.g. which is expressed only before birth). The protein may be aid transcription or translation gene or may regulate transcription or translation (e.g. a transcription factor or a protein that binds a transcription factor or polymerase). The protein may be a signalling molecule, such as an intracellular or extracellular signalling molecule (e.g. a hormone).

The protein may be an immune system gene, such as an antibody, T cell receptor, MHC molecule, cytokine (e.g IL-1, IL-2, IL-3, IL-4, IL-5, IL-6, IL-7, IL-8, IL-10, IL-10, TNF-α, TNF-β, TGF-β, an interferon (e.g. IFN-α, IFN-β, IFN-γ), chemokine (e.g. MIP-1α, MIP-1β, RANTES), an immune receptor (e.g. a receptor for a cytokine, interferon or chemokine, such as receptor for any of the above-mentioned cytokines, interferons or chemokines), a cell surface marker (e.g. macrophage, T cell, B cell, NK cell or dendritic cell surfacemarker)(eg. CD 1, 2, 3, 4, 5, 6, 7, 8, 16, 18, 19, 28, 40, or 45; or a natural ligand thereof) or a complement gene.

The protein may be a trophic factor (e.g. BDNF, CNTF, NGF, IGF, GMF, aFGF, bFGF, VEGF, NT3, T5, HARP) or an apolipoprotein. The protein may be a tumour suppressor genes (e.g. p53, Rb, Rap1A, DCC or k-rev) or a suicide gene (thymidine kinase or cytosine deaminase). The protein may be an antibody. Antibodies may be intact molecules, or fragments thereof, such as Fa, F(ab′)₂, Fv, scFv, or single-chain antibodies which are capable of binding to epitopic determinants. The antibody may be “humanised”, in that, amino acids in the non-antigen binding regions may be replaced to cause the antibody to more closely resemble a human antibody, whilst retaining its original binding activity.

The polypeptide may have enzymatic activity. Typically the polypeptide will be useful for industrial purposes. For example, xylanases, enzymes involved in plant cell wall modification, can be useful in paper manufacture. Alternatively, the polypeptide may be useful for therapeutic purposes, for example, as anticoagulants, such as hirudin, which may be useful in a method of treatment of the human or animal body, or as an antigenic polypeptide for use as an edible or extractable vaccine.

In a preferred embodiment the polypeptide is an interferon. In a yet more preferred embodiment the polypeptide is IFN-g. IFN-g, also known as immune interferon, mediates many immune responses, for example antiviral, anti-proliferative and several immunoregulatory actions in response to viral and pathogen infections (Pestka and Langers 1987; Lewis et al, 1988, Sen and Lengyel 1992). The human ifnG gene encodes a mature protein of 143 amino acids and is glycosylated at positions Asn 25 and Asn 97 (Gray et al, 1982; James et al, 1995; Rinderknecht et al, 1984). However, unglycosylated E. coli-derived recombinant IFN-g shows the same spectrum of biological activities as the natural glycosylated human IFN-g (Arora and Khanna 1996; Vega et at. 1990; Vilcek 1990). The three-dimensional structure of human IFN-g has been determined using E. coli expressed recombinant protein (Ealick et al, 1991). Recombinant IFN-g has been extensively tested clinically and used for the treatment of many diseases and disorders, including granulomatous disease (Bemiller et al, 1995; Weening et al, 1995), rheumatoid arthritis (Cannon at al. 1990; Machold et al, 1992) and atopic dermatitis (Hanifin et al, 1993; Ellis et al, 1999). IFN-g is also useful as an adjuvant in the vaccination of immunocompromised humans (Jaffe and Herberman 1988). In view of these applications and for the purposes of various biochemical studies, human IFN-g has been recombinantly expressed in a variety of hosts such as E. coli (Gray et al, 1982; Vega et al, 1990; Arora and Khanna 1996) monkey COS-7 cells (Gray et al, 1982), Chinese hamster ovary cells (James et al, 1995), Sf9 insect cells (James et al, 1995) and transgenic mice (Dobrovolsky et al, 1993; James et al, 1995; Lagutin et al, 1999). IFN-g can thus be expressed in any suitable cell system or organism.

In a further embodiment the coding region of the polynucleotide of the invention encodes a fusion protein. A fusion protein is a single polypeptide comprising at least two contiguous amino acid sequences that are not naturally found joined together. Preferably, the fusion protein will contain three sequences that are not naturally found together, more preferably four, five or more sequences. Typically, at least one of the sequences represents the sequence of the polypeptide of interest, that is, it has the sequence of a polypeptide that is desirably expressed in a plastid. Polypeptides of interest include those which provide herbicide, insecticide or disease resistance, selectable or scorable markers, modifications or additions to metabolic or signalling pathways, or be of no relation to the plastid, cell or organism in which it is expressed, as described above. Preferably, the fusion protein will contain the sequences of two, three or more polypeptides of interest, which may be the same or different.

Typically, the recombinantly expressed fusion protein enjoys greater stability in the plastid compared to the recombinantly expressed individual polypeptide of interest. Preferably, the fusion protein accumulates to higher levels than the individual polypeptide of interest when recombinantly expressed in the plastid. Therefore, in a preferred embodiment the fusion protein comprises the amino acid sequence of a polypeptide of interest, fused to another amino acid sequence, which other amino acid sequence increases the plastidic accumulation of the expressed fusion protein compared to the plastidic accumulation of the individually expressed polypeptide of interest. In a more preferred embodiment the fusion protein increases accumulation by up to 10-fold compared to individually expressed polypeptide of interest. In a yet more preferred embodiment the increase is up to 100-fold, and in an even more preferred embodiment the increase is up to 500-fold. Most preferably the increase is up to 1000-fold.

In a further embodiment, at least one of the polypeptide sequences within the fusion protein provides the fusion protein with a selectable or scorable property. This property aides in the purification of the fusion protein by allowing rapid and easy identification of fractions containing the fusion protein. The preferred sequences providing scorable properties include GUS, GFP (Meyer and Chilkoti, 2000). Most preferably the sequence is the sequence of GUS, or a biologically active variant thereof.

In a further embodiment the fusion protein comprises at least one but not more than 100 amino acid sequence that allows for the fusion protein to be readily purified. Typically, the number of purification sequences is not more than 5, more typically not more than 2, most typically 1. Typically such a sequence enables the fusion protein to be purified by affinity based methods. In a preferred embodiment, the purification sequence is a His-Tag. Typically the His-tag comprises multiple contiguous histidine residues, preferably from 3 to 20, more preferably from 4 to 10 most preferably 6 Typically the His-tag will be positioned at either or both of the N- and C-terminals of the fusion protein.

In a further embodiment, the polypeptide of interest is joined to other sequences by a sequence that can be cleaved to release the polypeptide of interest with substantially the same biological activity, or substantially the same amino acid sequence as the individually expressed protein of interest. In a preferred embodiment the cleavage sequence is IEGR, which is recognised and cleaved by Factor Xa (Nagi et al, 1985, Quinlan et al, 1989, Weame 1990).

In a particularly preferred embodiment, the polypeptide of interest is IFN-g. More preferably, the polypeptide of interest is IFN-g and is fused to the sequence of GUS. Yet more preferably the fusion protein comprises IFN-g, fused by an IEGR cleavage site, to a GUS sequence, and most preferably further comprises a His-Tag sequence.

The coding region may include a region encoding a signal sequence capable of targeting the encoded polypeptide to specific locations within the plastid. Typically the signal sequence can target the polypeptide to the inner or outer membrane of a chloroplast, to the stroma, to the inter-membrane space, to the thylakoid membranes or the compartments within the thylakoids. Heterologous sequence encoding such signal sequences may be derived from any suitable organism. Alternatively they can be generated from the coding region of signal sequences endogenous to the recipient plastome, by making conserved nucleotide substitutions in light of the degeneracy of the genetic code such that a heterologous coding sequence is generated encoding substantially the same polypeptide signal sequence as the endogenous coding sequence. In this context, a polypeptide signal sequence substantially the same may contain alterations of sequence, that is additions, deletions, insertions and/or inversions, as long as the targeting properties of the signal sequence are retained.

### Regulatory Regions

In one embodiment of the invention the heterologous region (b) further comprises at least one regulatory region, typically a plastomic regulatory region, operably linked to the coding region. Preferably the regulatory region is capable of securing expression of the coding sequence in the transplastome. The regulatory region is typically a promoter, enhancer or terminator, preferably a promoter in combination with an enhancer and/or terminator. A promoter is any nucleotide sequence capable of initiating transcription of a sequence 3′ (downstream) to it An enhancer is any nucleotide sequence capable of increasing the level of transcription initiating from a promoter and may act on a cis or trans basis. A terminator is any nucleotide sequence capable of promoting dissociation of RNA polymerase from the plastome. Regulatory regions may be derived from any organism suitable for the derivation of recipient plastomes and may be generated by recombinant techniques or synthetic means. Regulatory regions may be truncated or sequence alterations made, for example, in order to reduce or remove areas of homology to the proposed recipient plastome or to introduce or improve sequences essential for function as a regulatory region. Regulatory regions may be specifically designed de novo to ensure heterology to the recipient plastome and to comprise the essential features of functional regulatory regions. Typically, suitable promoter regions will contain well defined −10 and −35 sequence motifs (Tanaka et al, 1997; Isono et al, 1997; Kestermann et al, 1998). Typically a −10 sequence motif will have the sequence 5′-TATAAT-3′, and a −35 sequence motif will have the sequence motif 5′-TTGACA-3′, although the skilled person will appreciate that variants of these sequence can be used (Grierson and Covey, 1988). In this context, a −10 or −35 sequence motif variant is a sequence capable of binding to a plastid-encoded RNA polymerase in a manner which allows initiation of transcription at the intended site. The variant may differ from the typical motifs shown above by deletion, addition or substitution of 1, 2, 3 or more nucleotides in adjacent or non-adjacent positions as long as these properties are retained. In the −35 TTGACA hexamer, TTG is highly conserved. The distribution of bases among the chloroplast promoters are T (100%), T(84%), G(88%), A(80%), C(41%), A(62%). In the −10 TATAAT hexamer, TA-T is highly conversed. The distribution of bases are T(98%), A(92%), T(45%), A(60%), A(67%), T(93%). Thus, a skilled person will appreciate that some bases are more likely to vary than others. Typically the −10 sequence motif will be positioned 10 base pairs upstream of the transcription start site and the −35 sequence motif will be positioned 35 base pairs upstream of the transcription start site. However, the skilled person will appreciate that the precise positioning of these sequence motifs relative to each other and to the transcription start site is not critical to the function of the promoter. Promoters wherein the −10 and −35 sequence motifs show variation in position therefore also represent promoters of the invention. In this context, variation in position means that the −10 sequence motif will preferably be from 1 to 20, more preferably 5 to 15 bases upstream of the transcription initiation site, whereas the −35 sequence motif will preferably be from 25 to 45, more preferably 30 to 40 bases upstream of the transcription initiation site. Furthermore, the −10 and −35 motifs will preferably be separated by from between 1 to 20 bases, preferably by from between 16 to 20 bases. In a preferred embodiment the −10 sequence motif is positioned from 8 to 10 bases upstream of the transcription start site. In another preferred embodiment the −35 sequence motif is positioned from 32 to 35 bases upstream of the transcription start site. In yet another preferred embodiment the −10 and −35 sequence motifs are separated by from 16 to 20 bases. In the most preferred embodiment, the −10 sequence motif is positioned 10 bases upstream of the transcription start site, the −35 sequence motif is positioned 35 upstream of the transcription start site and the −10 and 35 sequence motifs are separated by 17 bases (Kung and Lin, 1985). In one embodiment the promoter provides constitutive transcription of the coding region. In another embodiment the promoter is inducible, and provides regulated transcription of the coding region. Preferably the expression of the coding region can be modulated by external stimuli. Examples of inducible promoters known in the art include those modulated by exposure to tetracycline, ecdysteroids, glucocorticoids, plant growth regulators such as abscisic acid, animal hormones, nitrates, metal ions (such as copper), environmental conditions (such as cold, heat, light or dark) and wounding. Furthermore, promoters may be used that are limited to expression in specific tissue or cell types, and/or during specific developmental stages, such as during flowering or senescence.

### Transforming Polynucleotides with Homologous Regulatory Regions

In another embodiment of the invention, a homologous flanking region of the transforming polynucleotide comprises a regulatory region, or a part of a regulatory region, homolgous to a sequence endogenous to the recipient plastome. During homologous recombination, regions in the recipient plastome are swapped for the homologous flanking regions of the transforming polynucleotide. The use of a regulatory region endogenous to the recipient plastome in a homologous flanking region of the transforming polynucleotide does not, therefore, cause sequence duplication in the transplastome. Thus, internal homologous recombination is not promoted and the transplastome is stable. In a preferred embodiment, the regulatory region is a promoter as defined above and is operably linked to the coding region of the heterologous region (b) such that expression of the coding region is secured in the transplastome. Additionally or alternatively, a homologous flanking region may comprise an enhancer sequence as defined above, capable of altering the rate of expression in the coding region in the heterologous region (b) of the transforming polynucleotide. In another embodiment, the 3′ homologous flanking region (c) of the transforming polynucleotide may comprise a terminator region, as defined above, operably linked to the coding region, that is homologous to a terminator region endogenous to the recipient plastome.

It should be understood that these particular embodiments do not preclude the presence of further regulatory regions in the heterologous region (b) as described above. In fact, a further embodiment of the invention anticipates the use of transforming constructs wherein regulatory regions are present in both the homologous flanking regions (a) and (c) and in the heterologous region (b) and these may contribute to the expression of the same or different coding sequences within the heterologous region (b). Similarly, there is also provided transforming polynucleotides wherein a single coding region has, for example, two promoters. In some cases (e.g. with double cauliflower mosaic virus 35S (CAMV35S)) this may improve expression.

### Transforming Polynucleotides with no Regulatory Regions

In another embodiment of the invention the transforming polynucleotide is provided with no regulatory region but is capable of securing expression of its coding region when integrated into the recipient plastome. Since regulatory regions are not present, duplication of endogenous sequences can be avoided in the transplastome, thus internal homologous recombination is not promoted and the transplastome remains stable. In the transforming polynucleotide provided by this embodiment the invention, a homologous flanking region is homologous to a region of the recipient plastome comprising part of a gene. The 5′ homologous flanking region of the transforming polynucleotide is homologous to a region of the recipient plastome gene downstream of its promoter. Thus, following integration of the transforming polynucleotide into the recipient plastome, the promoter of the pre-existing endogenous gene drives expression of the integrated transforming polynucleotide. The transforming polynucleotide is designed such that, following integration, the promoter of the gene is operably linked to the coding region of the heterologous region (b) of the transforming polynucleotide such that expression of the coding region is secured in the transplastome.

Additionally or alternatively, a homologous flanking region of the transforming polynucleotide may be homologous to a region of the recipient plastome comprising at least a part of the same or a different gene. Preferably the 3′ homologous flanking region of the transforming polynucleotide will be homologous to a region of a gene that is upstream of a terminator sequence. More preferably, following integration of the transforming polynucleotide, the coding region of heterologous region (b) of the transforming polynucleotide is operably linked to the downstream endogenous terminator region.

It should be understood that these particular embodiments do not preclude the presence of regulatory regions in the homologous flanking regions (a) and (c) or in the heterologous region (b) as described above. In fact, a further embodiment of the invention provides for the generation of tranplastomes wherein a coding sequence in the heterologous region (b) is operably linked to regulatory regions present in any or all of the recipient plastome, the homologous flanking regions (a) and (c), and/or the heterologous region (b). Furthermore, these regulatory regions may contribute to the expression of the same or different coding sequences within the heterologous region (b).

### Generation of Transforming Polynucleotides

Transforming polynucleotides of the invention may comprise DNA or RNA, preferably DNA. They may also include within them synthetic or modified nucleotides. The invention further provides double stranded polynucleotides comprising a polynucleotide of the invention and its complement.

Transforming polynucleotides of the invention may be produced recombinantly, synthetically, or by any means available to those of skill in the art. The polynucleotides are typically provided in isolated and/or purified form.

Although in general the techniques mentioned herein are well known in the art, reference may be made in particular to Sambrook et al, 1989, Molecular Cloning: a laboratory manual.

### Variants

Variants of regions of the transforming polynucleotides of the invention, in particular, promoter, enhancer and terminator regions and coding regions, may be obtained and used in the invention. This may be useful where, for example, sequence alterations can be used to alter homology with endogenous plastomic sequences of the recipient plastome, or to alter the functionality of the sequence within the plastome. Other sequence changes may be desired, for example, in order introduce restriction enzyme recognition sites. Variants may be isolated from natural sources or generated from existing sequences by site directed mutagenesis, synthesis of novel sequences or recombinant techniques. Naturally occurring variants may be obtained by probing cDNA or plastomic libraries with degenerate probes at medium to high stringency (for example 0.03M sodium chloride and 0.03M sodium citrate at from about 50° C. to about 60° C.). Alternatively, variants may be obtained using degenerate PCR.

Typically variants have at least 50% homology to the sequence from which they are derived, more typically at least 70%. Preferably variants have at least 90%, more preferably at least 95%, yet more preferably 99% homology to the sequence from which they are derived.

The term variant, as used herein, refers to a polynucleotide sequence that is altered by one or more nucleotide residues but retains the biological activity of the sequence from which it was derived. In this context, variants of regulatory regions, such as terminators, enhancers or promoters, will retain the activity of the sequence from which the variant was derived, that is, a promoter variant will retain the ability to initiate transgene expression, an enhancer variant will substantially retain the ability to promote transgene expression, whereas a terminator variant will retain the ability to promote dissociation RNA polymerase from the transgene and thus terminate transgene expression. Variants of coding sequences will retain the ability to encode a polypeptide having substantially the same biological activity as a polypeptide encoded by a naturally occurring coding sequence. In this context, biological activity refers to the binding specificity, enzymatic, structural and immunological properties of the naturally occurring polypeptide. In a preferred embodiment a biologically active variant of a polypeptide will retain substantially the same binding specificity or enzymatic properties as the naturally occurring polypeptide. In a more preferred embodiment the biologically active variant of a polypeptide will retain substantially the same binding specificity as the naturally occurring polypeptide.

Alterations may include additions, insertions, deletions, substitutions or inversions. The terms addition or insertion, as used herein, refer to a change in the polynucleotide sequence resulting in the addition of one or more nucleotide residues as compared to the naturally occurring molecule. Preferably the number of nucleotide additions or insertions will be at most 40, more preferably at most 20, yet more preferably at most 10, and most preferably at most 5. The term deletion, as used herein, refers to a polynucleotide sequence wherein one or more nucleotide residues are absent as compared to the naturally occurring molecule. Preferably the number of nucleotide deletions will be at most 40, more preferably at most 20, yet more preferably at most 10, and most preferably at most 5. The term substitution, as used herein, refers to the replacement of one or more nucleotide residues by different residues. Preferably the number of nucleotide substitutions will be at most 40, more preferably at most 20, yet more preferably at most 10, and most preferably at most 5. The term inversion, as used herein, refers to a polynucleotide sequence wherein a contiguous region within the sequence is reversed in orientation relative to the remaining molecule. Preferably the number of contiguous regions of sequence inverted will be 4, more preferably 3, yet more preferably 2, most preferably 1.

### Testing New Transforming Polynucleotides

Testing may be achieved, for example, by their substitution in place of an equivalent regulatory region of a suitable vector as described below, typically pVSR 326, transformation of a plastid of a plant cell, and selection of the transformed cell. Regulatory sequence function can be tested by transgene expression analysis of the transformed cell. Subsequent generation of a callus, a transplastomic plant and its progeny allow suitable analysis transplastome stability using a protocol as exemplified below. The skilled person will appreciate that the choice of probe is dependant on the sequence of the transforming polynucleotide used, and can be readily derived from the transforming polynucleotide itself. Testing of maternal inheritance can be performed by transgene expression analysis of wild type plants pollinated by transplastomic plants of the invention.

Of particular relevance to such tests, the transforming polynucleotide of the invention, or a fragment thereof, may be used to produce a primer, e.g. a PCR primer for expression analysis by RT-PCR or plastomic analysis for the presence of the integrated transgene, a primer for an alternative amplification reaction, or a probe e.g. labeled with a revealing label such as ³²P or ³⁵S, enzyme labels, or other protein labels such as biotin for northern and Southern analysis of transplastomic cells, calli and plants. Such primers, probes and other fragments will preferably be at least 10, preferably at least 15 or 20, for example at least 25, 30 or 40 nucleotides in length.

### Cloning Vectors

To facilitate cloning of the transforming polynucleotide of the invention, it can be incorporated into recombinant replicable vectors. Such vectors may be used to replicate the nucleic acid in a compatible host cell. Thus in a further embodiment, the invention provides a method of making transforming polynucleotides of the invention by introducing a transforming polynucleotide of the invention into a replicable vector, introducing the vector into a compatible host cell, and cultivating the host cell under conditions which bring about replication of the vector. The vector may be recovered from the host cell. Suitable host cells are described below in connection with expression vectors. Bacterial cells, especially E. coli are preferred.

The vectors may be for example, plasmid, cosmid, virus or phage vectors provided with an origin of replication. The vectors may contain one or more selectable marker genes, for example antibiotic ampicillin resistance genes. These will generally be operably linked to regulatory sequences capable of securing their expression in the host cell, as described herein for the coding sequences of the invention. The vector may contain one scorable marker, as described above, preferably interrupted by the integration of the transforming polynucleotide, to allow cells transformed by recombinant vectors (i.e. comprising the transforming polynucleotide) to be discriminated from those transformed with non-recombinant vector.

### Transforming Vectors

In one embodiment of the invention, a vector suitable for use in the generation of transplastomic cells is provided, wherein the vector comprises:

(a) a 5′ sequence homologous to a region of the recipient plastome comprising a unique restriction site at its 5′ terminus; and

(b) joined at via a unique restriction site to;

(c) a 3′ sequence homologous to a region of the recipient plastome comprising a unique restriction site at its 3′ terminus.

The term “unique restriction site” refers to a restriction site present only once in that particular vector. Exposure to the relevant enzyme, therefore, causes the vector to be restricted in one place only. For example, restriction of the unique site defined in (b) allows for the insertion of a heterologous sequence of choice. Alternatively restriction at sites 5′ and 3′ to a particular homologous flanking region allow for convenient deletion of this region, and insertion of new polynucleotide sequences. Such strategies may be useful to enable, for example, to modify the transforming polynucleotide as appropriate or to enable testing of putative regions for new transforming polynucleotides of the invention. Testing may be, for example, for the purposes of studying the effect of using regions, such as homologous flanking regions or regulatory regions, with different sequences, on transgene expression, transplastome stability or maternal inheritance. Suitable methods for these tests are discussed above.

Typically a heterologous sequence is inserted into the restriction site defined in (b) above. The heterologous sequence is heterologous to the recipient plastome and comprises:

(1) a coding region for at least one selectable or scorable marker operably linked to regulatory regions capable of securing expression of the coding sequence in the transplastome, wherein each regulatory region and each coding region comprises a unique restriction sites at its 5′ and 3′ borders; and optionally

(2) regulatory regions capable of securing expression of a coding sequence in the transplastome, wherein each regulatory region comprises unique restriction sites at 5′ and 3′ borders; and optionally

(3) a coding sequence operably linked to the regulatory regions of (2).

In a preferred embodiment, a heterologous sequence inserted into the restriction site defined in (b) above comprises:

(i) a promoter region functional in the transplastome joined by a unique 5′ restriction site to the homologous sequence defined in (a) above and operably linked by a unique 3′ restriction site to;

(ii) a coding region encoding a selectable or scorable marker, and operably linked by a unique restriction site to;

(iii) a terminator region; and joined ; and joined by a unique restriction site to;

(iv) a promoter region functional in the transplastome and operably linked by a unique restriction site to;

(v) a coding region encoding a selectable or scorable marker, and operably linked by a unique restriction site to;

(vi) a terminator region, joined by a unique restriction site to the sequence defined in (c) above.

Typically, the vector is suitable for use in the transformation of tobacco plastids, more preferably tobacco chloroplasts. Preferably the promoter defined in (i) or (iv) is derived from the rice psbA gene promoter or the rice rrn gene promoter. Preferably the terminator defined in (iii) or (vi) is derived from the 3′ untranslated region of the rice psbA gene or 3′ untranslated region of the rice rbcL gene. Preferably the selectable or scorable marker defined in (ii) or (v) is derived from the coding sequence of the aadA or uidA genes. In the most preferred embodiment, the vector is pVSR 326 as exemplified below.

### Cells for Transformation

The cell used for transformation may be from any suitable organism (see above list) and may be in any form. For example, it may be an isolated cell, e.g. a protoplast or single cell organism, or it may be part of a plant tissue, e.g. a callus, for example a solid or liquid callus culture, or a tissue excised from a plant, or it may be part of a whole plant. It may, for example, be part of an embryo, or a meristem, e.g. an apical meristem of a shoot. Preferably the cell is a cell containing chloroplasts, e.g. a leaf or stem cell, most preferably a leaf cell derived from the abaxial side of the leaf.

Transformation may thus give rise to a chimeric tissue or plant in which some cells are transgenic and some are not.

### Transformation Techniques

Generation of the transplastome is brought about by the insertion of the polynucleotide defined above. The polynucleotide may be inserted by any method known in the art, such as recombinant techniques, random insertion, or site directed integration. Preferably the method of polynucleotide insertion is site directed integration, more preferably by the process of homologous recombination. The transforming polynucleotide may be inserted into an isolated plastome or an in vivo plastome within a plastid. The plastid used may be in vivo or ex vivo. Insertion of the transforming polynucleotide is preferably performed by transformation of an in vivo plastid. Preferably, the plastid is within a cell, though it may be in isolated form.

Cell transformation may be achieved by any suitable transformation method, for example the transformation techniques described herein. Preferred transformation techniques include electroporation of plant protoplasts (Taylor and Walbot, 1985), PEG-based procedures (Golds et al, 1993), microinjection (Neuhas et al, 1987; Potrykus et al, 1985), injection by galinstan expansion femtosyringe (Knoblauch et al, 1999) and particle bombardment (Boynton et al, 1988; Svab et al, 1990; Svab and Maliga 1993; U.S. Pat. No. 5,451,513; U.S. Pat. No. 5,545,817; U.S. Pat. No. 5,545,818; U.S. Pat. No. 5,576,198; U.S. Pat. No. 5,866,421). Particle bombardment is particularly preferred.

### Selection of Transformed Cells and Generation of Homotransplastomic Cells

Homotransplastomic (see above) plastids, cells, plants, seeds, plant parts, plant tissues are preferred.

Cells generated by the transformation techniques discussed above will typically be present in chimeric tissues, and thus will be surrounded by other non-transformed cells. Furthermore, due to the multiple genome copies within each plastid, transplastomic plastids will typically contain multiple copies of untransformed plastomes. In order to produce homotransplastomic cells, that is cells in which all plastids are homotransplastomic, in that all genomes within those plastids comprise the transforming polynucleotide of the invention, it is necessary to undergo rounds of screening. Screening will be carried out via an expressed selectable or scorable marker coding region, as defined above, in the integrated polynucleotide. Preferred selectable markers include the aadA gene or the NPTII gene.

Homotransplastomic cells can be generated by mutiple rounds of screening of the primary transformed cells for the presence of the selectable or scorable marker. Preferably, at least one round of screening is used, more preferably at least two rounds, most preferably three rounds or more. Typically the homotransplastomic nature of the thus generated cells are ascertained. Homotransplastomicity can be assayed by analysis of isolated plastomic DNA by Southern analysis or by performing polymerase chain reaction amplification. These techniques are suitably sensitive such that the presence of a single untransformed plastome could be detected.

### Generating Stable Transplastomic Plants and Seeds

Transplastomic or homotransplastomic cells may be regenerated into a transgenic plant by techniques known in the art. These may involve the use of plant growth substances such as auxins, giberellins and/or cytokinins to stimulate the growth and/or division of the transplastomic or homotransplastomic cell. Similarly, techniques such as somatic embryogenesis and meristem culture may be used. Regeneration techniques are well known in the art and examples can be found in, e.g. U.S. Pat. No. 4,459,355, U.S. Pat. No. 4,536,475, U.S. Pat. No. 5,464,763, U.S. Pat. No. 5,177,010, U.S. Pat. No. 5,187,073, EP 267,159, EP 604,662, EP 672,752, U.S. Pat. No. 4,945,050, U.S. Pat. No. 5,036,006, U.S. Pat. No. 5,100,792, U.S. Pat. No. 5,371,014, U.S. Pat. No. 5,478,744, U.S. Pat. No. 5,179,022, U.S. Pat. No. 5,565,346, U.S. Pat. No. 5,484,956, U.S. Pat. No. 5,508,468, U.S. Pat. No. 5,538,877, U.S. Pat. No. 5,554,798, U.S. Pat. No. 5,489,520, U.S. Pat. No. 5,510,318, U.S. Pat. No. 5,204,253, U.S. Pat. No. 5,405,765, EP 442,174, EP 486,233, EP 486,234, EP 539,563, EP 674,725, WO91/02071, WO 95/06128 and WO 97/32977.

In many such techniques, one step is the formation of a callus, i.e. a plant tissue comprising expanding and/or dividing cells. Such calli are a further aspect of the invention as are other types of plant cell cultures and plant parts. Thus, for example, the invention provides transplastomic or homotransplastomic plant tissues and parts, including embryos, meristems, seeds, shoots, roots, stems, leaves and flower parts. These may be chimeric in the sense that some of their cells are transplastomic or homotransplastomic and some are not. Similarly they may be chimeric in the sense that all cells are transplastomic but only some are homotransplastomic.

Regeneration procedures will typically involve the selection of transplastomic and/or homotransplastomic cells by means of marker genes, as discussed above. The regeneration step gives rise to a first generation transplastomic or homotransplastomic plant. The invention also provides methods of obtaining transplastomic or homotransplastomic plants of further generations from this first generation plant. These are known as progeny transplastomic or homotransplastomic plants. Progeny plants of second, third, fourth, fifth, sixth and further generations may be obtained from the first generation transplastomic or homotransplastomic plant by any means known in the art.

Thus, the invention provides a method of obtaining a transplastomic or homotransplastomic progeny plant comprising obtaining a second-generation transplastomic or homotransplastomic progeny plant from a first-generation transplastomic or homotransplastomic plant of the invention, and optionally obtaining transplastomic or homotransplastomic plants of one or more further generations from the second-generation progeny plant thus obtained.

Such progeny plants are desirable because the first generation plant may not have all the characteristics required for cultivation. For example, for the production of first generation transgenic plants, a plant of a taxon that is easy to transform and regenerate may be chosen. It may therefore be necessary to introduce further characteristics in one or more subsequent generations of progeny plants before a transplastomic or homotransplastomic plant more suitable for cultivation is produced.

Progeny plants may be produced from their predecessors of earlier generations by any known technique. In particular, progeny plants may be produced by:

obtaining a transplastomic or homotransplatomic seed from a transplastomic or homotransplastomic plant of the invention belonging to a previous generation, then obtaining a transplastomic or homotransplastomic progeny plant of the invention belonging to a new generation by growing up the transplastomic or homotransplastomic seed; and/or

propagating clonally a transplastomic or homotransplastomic plant of the invention belonging to a previous generation to give a transplastomic or homotransplastomic progeny plant of the invention belonging to a new generation; and/or

crossing a first-generation transplastomic or homotransplastomic plant of the invention belonging to a previous generation with another compatible plant to give a transplastomic or homotransplastomic progeny plant of the invention belonging to a new generation; and optionally

obtaining transplastomic or homotransplastomic progeny plants of one or more further generations from the progeny plant thus obtained.

These techniques may be used in any combination. For example, clonal propagation and sexual propagation may be used at different points in a process that gives rise to a transplastomic or homotransplastomic plant suitable for cultivation. In particular, repetitive back-crossing with a plant taxon with agronomically desirable characteristics may be undertaken. Further steps of removing cells from a plant and regenerating new plants therefrom may also be carried out.

Also, further desirable characteristics may be introduced by transforming the cells, plant tissues, plants or seeds, at any suitable stage in the above process, to introduce desirable coding sequences other than the polynucleotides of the invention. This may be carried out by conventional breeding techniques, e.g. fertilizing a transplastomic or homotransplatomic plant of the invention with pollen from a plant with the desired additional characteristic. Alternatively, the characteristic can be added by further transformation of the plant obtained by the method of the invention, using the techniques described herein for further plastomic transformation, or by nuclear transformation using techniques well known in the art such as electroporation of plant protoplasts, transformation by Agrobacterium tumefaciens or particle bombardment. Particle bombardment is particularly preferred for nuclear transformation of monocot cells. Preferably, different transgenes are linked to different selectable of scorable markers to allow selection for both the presence of further transgenes. Selection, regeneration and breeding techniques for nuclear transformed plants are known in the art. Techniques along the lines of those described may be used.

### Use of Transplastomic Plants

The invention also provides methods of obtaining crop products by harvesting, and optionally processing further, transplastomic or homotransplastomic cells, calli, plants or seeds of the invention. By crop product is meant any useful product obtainable from a crop plant.

Such a product may be obtainable directly by harvesting or indirectly, by harvesting and further processing. Directly obtainable products include: grains, e.g. grains of monocotyledonous species, preferably graminaceous species, for example wheat, oats, rye, rice, maize, sorghum, triticale, especially wheat; other seeds; shoots, especially tubers, such as potato tubers; fruit; and other plant parts, for example as defined herein. Alternatively, such a product may be obtainable indirectly, by harvesting and further processing. Examples of products obtainable by further processing are: flour; oil; rubber; beverages such as juices and fermented and/or distilled alcoholic beverages; food products made from directly obtained or further processed material, e.g. bread made from flour or margarine made from oil; tobacco and tobacco products such as cigarettes and cigars; fibres, e.g. cotton, linen, flax and hemp fibres and textile items made therefrom; paper or timber derived from woody plants.

### Purification of Transplastomically-expressed Polypeptides

The protein may be purified by any method available in the art. Typically, the protein purified is a fusion protein. Typically, the total protein content of the cell or organism is extracted and the fusion protein isolated by affinity based methods suitable to the sequence of the fusion protein.

In a preferred embodiment, prior to affinity purification the fusion protein is purified by fractionation. Fractionation can be performed using any method known in the art and typically separates proteins based on their physical properties, for example, their size, mass, hydrophobicity or hydrophilicity. Fractionation thus results in the separation of the total protein extract into a number of discreet fractions. The fraction (or fractions) containing the fusion protein can then be identified and further purification limited the fraction (or fractions) so identified. Identification can be performed by any method known in the art, although in a preferred embodiment, the fuision protein comprises a scorable property to allow rapid identification of the fraction. In the most preferred embodiment, the fusion protein comprises the sequence of GUS, and the fraction (or fractions) containing the fusion protein can thus be detected by histochemical assays and measured by fluorometric assays (Gallagher, 1992). Fractionation may be performed any number of times, and each fractionation may separate the proteins based on the same or different properties as the previous fractionation(s). Typically, fractionation is performed no more than 5 times, more typically no more than 3 times, most typically once. Following fractionation, the fusion protein can be further purified from the fraction (or fractions) identified as containing the fusion protein by any method known in the art

Typically the method of further purification used is an affinity based method, and is particular to the fusion protein. In a preferred embodiment, the fusion protein comprises a His-tag and affinity purification is performed using a Ni-NTA agarose column under suitable conditions. Thus, the fusion protein is retained in the affinity column whilst other proteins present in the fraction are washed out, and the conditions in the column are then altered leading to the release of the fusion protein, which can thus be collected in a purified form.

Fusion proteins thus purified can be further processed in order to release the plypeptide of interest, typically by cleavage of the fusion protein. Cleavage may be performed by any method known in the art, and the method chosen will be particular to the cleavage sequence (or sequences) present in the fusion protein. In a preferred embodiment the cleavage sequence is an Fa-Xa site, IEGR, and is cleaved by incubation with factor Xa.

The resultant mixture thus contains the polypeptide of interest and polypeptide fragments representing the rest of the fusion protein. The polypeptide of interest can then be isolated by any method known in the art, for example either affinity based methods or fractionation. In a preferred embodiment, the amino acid sequence recognised and bound by the affinity column is present in the rest of the fusion protein fragment. Thus applying the resultant mixture to the affinity column a further time leads to the elution of the polypeptide of interest, whilst the rest of the fusion protein is retained in the column. The elute thus represents the purified polypeptide of intercest. The purity and identity of the purified polypeptide of interest can then be verified by any method known in the art, for example, by western blotting, by sequencing, or by assaying the putative properties, enzymatic or otherwise, of the polypeptide.

### EXAMPLES

In the experimental details which follow, weights are given in grams (g), milligrams (mg) or micrograms (μg), all temperatures are given in degrees centigrade (° C.), concentrations are given as molar (M), millimolar (mM) or micromolar (mM), nanomolar (μM), picomolar (μM) and volumes are given in litres (L), millilitres (ml), microlitres (μl), unless otherwise indicated.

Abbreviations: Base pairs (bp),

### Example 1

### Generating the Plastid Transformation Vector (pVSR326)

Polymerase chain reaction (PCR) was performed to amplify defined DNA fragments through appropriate primers (Sarkar and Sommer 1990). Convenient restriction sites were introduced into primers for easy cloning. Standard procedures were followed for cloning the PCR products (Sambrook et al, 1989).

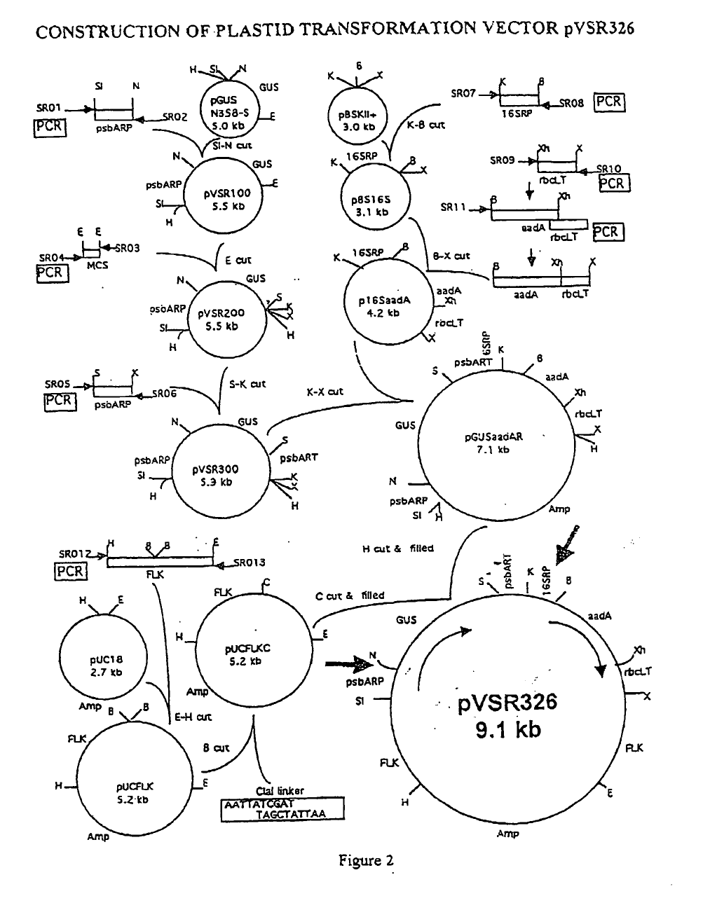

The plastid transformation vector, pVSR326 (FIG. 2), was constructed using the rrn and psbA promoters and 3′ untranslated regions of psbA and rbcL genes from rice plastome primary clones (Hiratsuka et al, 1988). The selectable aadA and reporter uidA genes were cloned from pUC-atpX-AAD (Goldschmidt-Clermont 1991) and pGUSN358-S (Clontech) plasmids, respectively. The tobacco plastid genome sequences spanning rbcL-accD genes (Shimozaki et al, 1986) were used for site specific integration of chimeric aadA and uidA genes into plastid DNA.

A 480 bp fragment of psbA gene promoter, psbARP, (SEQ ID NO: 25, nucleotides 1615-1164, EMBL Acc. No. X15901) was PCR amplified using pRB7 template DNA and SR01 (SEQ ID NO: 1)—SR02 (SEQ ID NO: 2) primer combination. All subsequent PCR reactions were carried out in a 50 μl volume using 10 ng of DNA template, 0.2 mM INTP's, 100 pmoles of each primer and Pfu polymerase (Stratagene, USA). The reaction was carried out for 25 cycles, each cycle being 30 sec at 94° C., 30 sec at 50° C. and 2 min 72° C. The resulting DNA was digested with restriction endonucleases SalI-NcoI and inserted upstream of the uidA gene in the plasmid pGUSN358-S (Clontech, USA) to create pVSR100 intermediate vector (FIG. 2). A multiple cloning site (MCS) was introduced into pVSR100 using SR03 (SEQ ID NO: 3) —SR04 (SEQ ID NO: 4) primers. The SR03 and SR04 primers are complementary to each other and provide cohesive ends that are compatible to EcoRI digested pVSR100 vector. The SR03 and SR04 oligos were designed in such a way that the EcoRI site is not recreated upon ligation in the vector. The resulting plasmid was named as pVSR200. A 374 bp 3′ end of psbA gene, psbART, (SEQ ID NO: 27, nucleotides 81-134233 EMBL Acc. No. X15901) fragment was amplified using pRB7 template DNA and primers SR05 (SEQ ID NO: 5) and SR06 (SEQ ID NO: 6) The amplified 3′ end of psbA gene fragment was digested with SacI-KpnI and cloned into pVSR200 to create pVSR300.

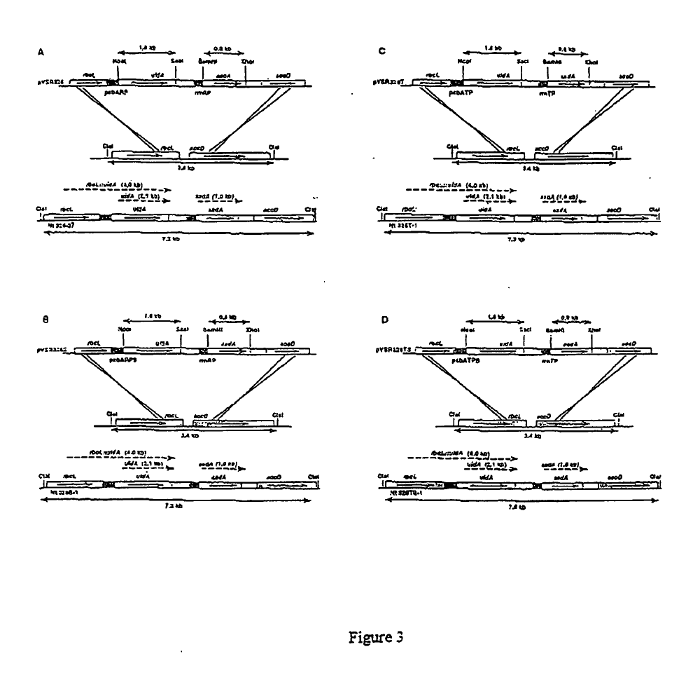

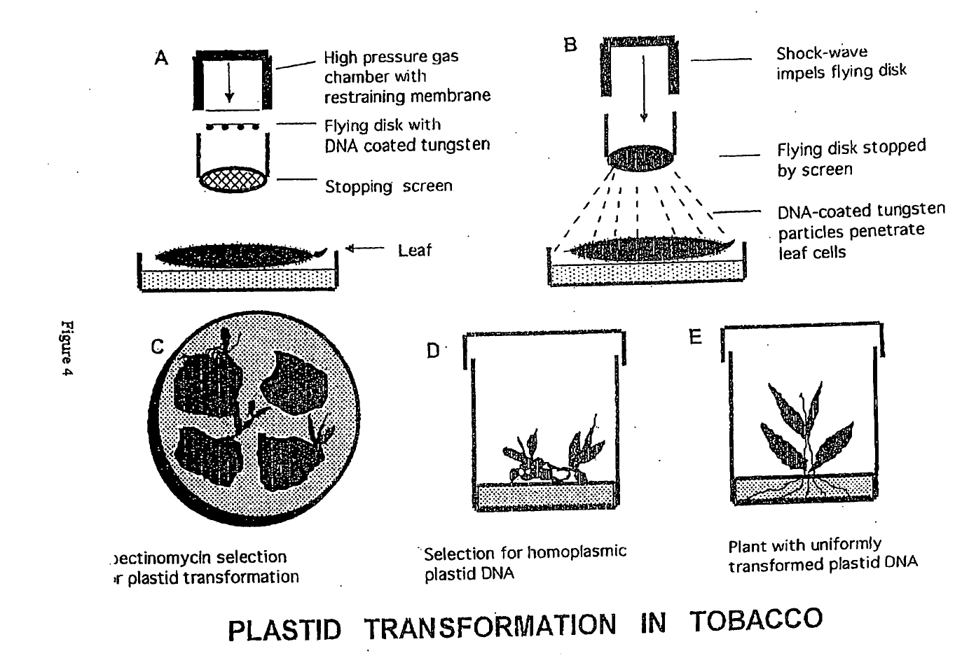

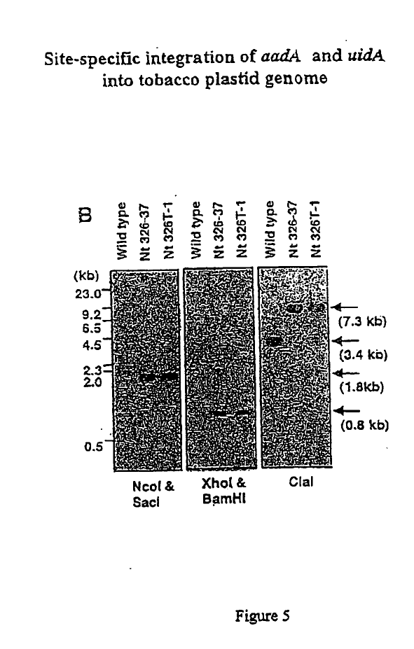

A 117 bp fragment (SEQ ID NO: 28, nucleotides 91,100-91,216, EMBL Acc. No. X15901) of 16S rRNA operon promoter, (16SRP) was amplified using pRP7 template and primers SR07 (SEQ ID NO: 7) and SR08 (SEQ ID NO: 8). The amplified DNA was digested with KpnI-BamHI and cloned into pBluescript II SK+ (STRATAGENE, USA) vector to create pBS16S. A 256 bp fragment of 3′ end of rbcL gene, rbcLRT, (SEQ ID NO: 29, nucleotides 55,529-55,784, EMBL Acc. No X15901) was amplified using pRPI template DNA and SR09 (SEQ ID NO: 9) and SR10 (SEQ ID NO: 10) primers. The amplified fragment, after gel purification, was used as primer in the “Megaprimer” method of PCR (Sarkar and Sommer 1990) and SR11 (SEQ ID NO: 11) primer as the other primer and pUc-atpX-AAD (Goldschmidt-Clermont 1991) as template DNA to amplify aadA coding region along with 3′ end of rbcL. The first 18 bases in SR11 primer are complimentary to the 3′ end of the aadA gene and the last 18 bases are complimentary to 3′ end of the rbcL gene. A XhoI restriction site has been introduced in between the aadA coding region and 3′ end of rbcLRT fragment to facilitate easy exchange of aadA coding region with any other gene of interest. The amplified product was digested with BamHI-XbaI and cloned into pBS16S vector in the same sites to create p16SaadA vector. The aadA chimeric gene was taken as KpnI-XbaI fragment from p16SaadA and cloned into pVSR300 vector in the same sites to create pGUSaadAR vector. A 2572 bp plastid targeting sequence (SEQ ID NO: 35, nucleotides 58,056-60,627; EMBL Acc. No. Z00044) was PCR amplified using SR12 (SEQ ID NO: 12) and SR13 (SEQ ID NO: 13) primers and pTB22 (Shinozaki et al, 1998) as template DNA. The targeting sequence was digested with EcoRI-HindIII and cloned into pUC18 in the same restriction sites to create pUCFLK plasmid. A XhoI site present in the targeting sequence (nucleotide 60,484; EMBL Acc. No. Z00044) has been removed through site directed mutagenesis in order to make XhoI site present between aadA coding region and 3′ end of rbcL transcription terminator as unique site in the final pVSR326 vector (FIG. 2 and FIG. 3A). Further, a ClaI site containing linker (SEQ ID NO: 40) has been inserted into pUCFLK in between BamHI sites (nucleotides 59,286 and 59,306; EMBL Acc. No. Z00044) to create pUCFLKC. The final plastid transformation vector, pVSR326, was created by introducing chimeric aadA and uidA containing sequences from pGUSaadAR as HindIII fragment at ClaI site of pUCFLKC after treating both the fragments with Klenow to generate blunt ends.

### Example 2

### Generating Plastid Transformation Vector (pVSR 326S)

The pVSR326S (FIG. 3B) was derived from pVSR326 vector (FIG. 2) by replacing the rice psbA promoter with the truncated (203 bp) rice psbA promoter. The 203 bp fragment of psbA gene promoter, psbARPS, (SEQ ID NO: 26, nucleotides 1366-1164, EMBL Acc. No. X15901) was PCR amplified using pRB7 template DNA and SR21 (SEQ ID NO: 21)—SR02 (SEQ ID NO: 2) primer combination. The fragment was gel purified and cleaved with SphI-Nco I restriction enzymes and cloned in the vector pVSR326 in the same restriction sites after replacing the psbARP promoter.

### Example 3

### Generating the Plastid Transformation Vector (pVSR 326T)

The pVSR326T (FIG. 3C) was derived from pVSR326 vector (FIG. 2) by replacing the rice psbA promoter with the 484 bp tobacco psbA promoter. The 484 bp fragment of psbA gene promoter, psbATP, (SEQ ID NO: 30, nucleotides 2079-1596, EMBL Acc. No. Z00044) was PCR amplified using tobacco (Nicotiana tabacum cv. Petit Hawana) template DNA and SR22 (SEQ ID NO. 22)—SR23 (SEQ ID NO: 23) primer combination. The fragment was gel purified and cleaved with PstI-Nco I restriction enzymes and cloned in the vector pVSR326 in the same restriction sites after replacing the psbARP promoter to get an intermediate vector pVSR326T100. The 375 bp psba 3′ region (SEQ ID. NO: 38), nucleotides 533-159, EMBL Acc. No. Z00044, was PCR amplified using tobacco cv. Petit Hawana template DNA and SR36 (SEQ ID NO: 36)—SR37 (SEQ ID NO: 37) primer combination. The PCR amplified fragment was cloned, as a SacI-KpnI fragment, into the same restriction sites of pVSR326T100, after replacing psbART, to generate the vector pVSR326T.

### Example 4

### Generating the Plastid Transformation Vector pVSR 326TS

The pVSR326TS (FIG. 3D) was derived from pVSR326T vector (FIG. 2) by replacing the tobacco long psbA promoter with the 222 bp tobacco psbA promoter. The 222 bp fragment of psbA gene promoter, psbATPS, (SEQ ID NO.31, nucleotides 1817-1596, EMBL Acc. No. Z00044) was PCR amplified using tobacco cv. Petit Hawana template DNA and SR23 (SEQ ID NO. 23)—SR24 (SEQ ID NO: 24) primer combination. The fragment was gel purified and cleaved with PstI-Nco I restriction enzymes and cloned in the vector pVSR326 in the same restriction sites, after replacing the psbARP promoter, to generate the vector pVSR 326TS.

### Example 5

### Plastid Transformation and Plant Regeneration

Tobacco (Nicotiana tabacum cv. Petit Havana, obtained from Prof. P. Maliga) was transformed according to the method described by Svab and Maliga (1993) using Bio-Rad PDS1000 helium driven Biolistic gun. Typically, a tobacco leaf was placed on modified MS medium (Murashige and Skoog 1962) containing 0.1 mg/l thiamine, 100 mg/l inositol, 3% sucrose, 2 mg/l BA and 0.1 mg/l NAA, 0.6% agar, pH 5.8) and bombarded on the abaxial side by vector DNA coated on to tungsten particles (M17 Bio-Rad). Transformed shoots were selected on RMOP medium (MS medium supplemented with sucrose (30 g/l), thiamine HCl (1.0 mg/l), napthalene acetic acid (0.1 mg/l), benzyl aminopurine (1.0 mg/l), inositol (100 mg/l), pH 5.8; Svab et al, 1990) containing 500 mg/l spectinomycin dihydrochloride. Three additional cycles of regeneration on RMOP medium containing spectinomycin (500 mg/l) was carried out to obtain uniformly transformed plastids. Homoplasmic plants were grown to maturity in the green house.

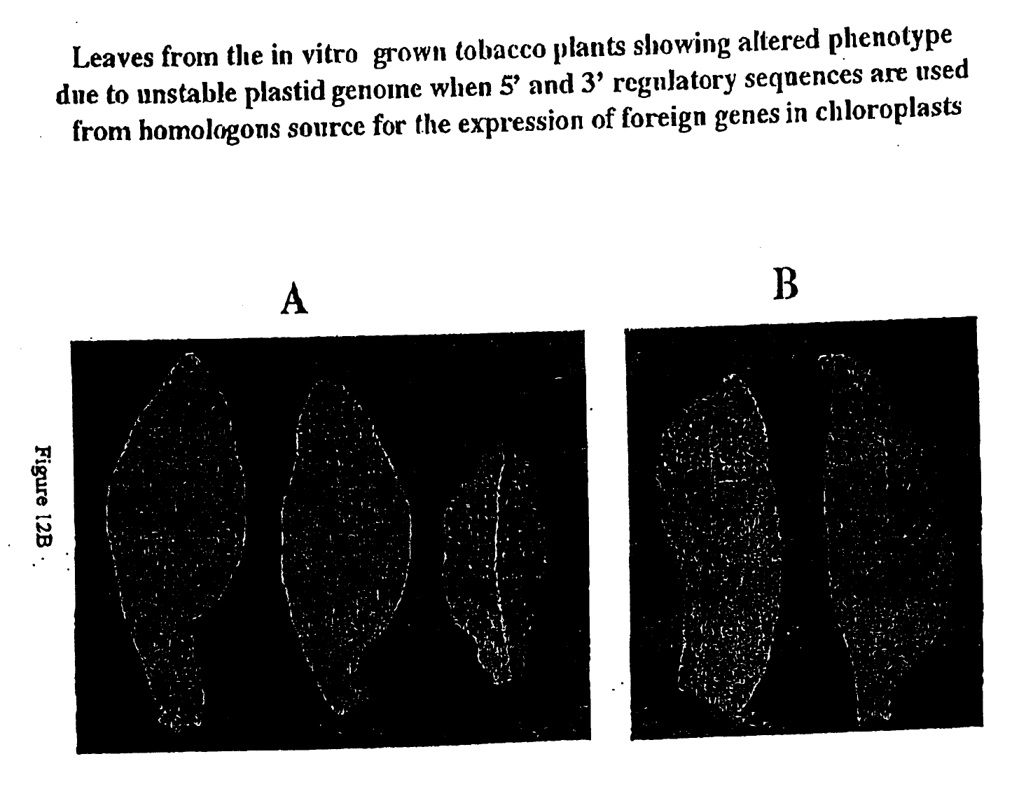

From 95 bombarded leaves, a total of 112 green plants were recovered by selection on RMOP medium containing spectinomycin (500 mg/L) for pVSR326 vector. Similarly using 22 pVSR326S-transformed, 23 pVSR326T-transformed and 28 pVSR 326TS-transformed green plants, were recovered from 20 bombarded leaves. PCR based preliminary screening confirmed the presence of uidA and aadA gene sequences in the total plant DNA of green shoots. Two additional cycles of regeneration under spectinomycin selection resulted in the production of homoplasmic transplastomic lines (plants with transformed plastid genome; nomenclature adopted after Maliga 1993) for the introduced DNA. Three plants transformed with pVSR 326T and four plants transformed with pVSR 326TS exhibited loss of chlorophyll pigment (FIG. 12B). Further analysis was then carried out on the homoplasmic transplastomic lines generated.

### Example 6

### Analysis of the Generated Transplastomic Lines for Site Specific Integration and Confirming Homotransplastomic Status

Total DNA isolated from transgenic and control tobacco plants (Mettler 1987) was digested with various restriction endonucleases (Gibco-BRL) as defined below, separated on 0.7% agarose gels and transferred to Hybond N+ membrane (Amersham) using the capillary method (Sambrook et al, 1989). The PCR generated and gel purified psbA (SEQ ID NO: 39),16S rRNA (SEQ ID NO: 32), uidA (SEQ ID NO: 33), aadA (SEQ ID NO: 34) and targeting sequence fragments (SEQ ID NO: 35) amplified using SR17 (SEQ ID NO: 17)—SR18 (SEQ ID NO: 18), SR19 (SEQ ID NO: 19)—SR20 (SEQ ID NO: 20), SR41 (SEQ ID NO: 41)—SR42 (SEQ ID NO: 42), SR43 (SEQ ID NO: 43)—SR44 (SEQ ID NO: 44) and SR12 (SEQ ID NO: 12)—SR13 (SEQ ID NO: 13) primer pairs, respectively, were used as probes. Hybridizations were carried out at 65° C. for 16 h with ³²P-labeled DNA probes generated by nick translation (Gibco-BRL, USA). The membranes were washed once for 15 min in 0.1×SSC, 0.1% SDS at 65° C. then twice for 15 min in 0.1×SSC, 0.1% SDS at 65° C. and subjected to autoradiography.

Total plant DNA samples isolated from putatively transformed lines (GUS positive in histochemical assay) were analyzed for the presence and site specific integration of aadA and uidA coding regions (FIG. 5). Southern hybridization experiments confirmed the stable and site specific integration of chimeric aadA and uidA genes into the chloroplast genome. When total plant DNA was digested with NcoI-SacI and probed with uidA coding region, a 1.8 kb fragment hybridized in transplastomic lines. Further analysis with aadA probe also confirmed the integration of chimeric genes into plastid genome. When total plant DNA was digested with BamHI-XhoI and probed with aadA coding region, the probe hybridized to a 0.8 kb fragment. When total plant DNA was digested with ClaI and probed with the targeting sequences (rbcL-accD), the probe hybridized to a 3.4 kb fragment in wild type plant and a 7.3 kb fragment in transplastomic lines, indicating the site specific integration of the chimeric genes. The absence of a 3.4 kb fragment in transplastomic lines is clear evidence of the homoplasmic nature of the plastid genome after transformation. There were no nonspecific bands in any of the transplastomic lines when hybridized with either aadA, uidA or plastid targeting sequence (rbcL-accD) probes which is direct evidence for the integrity of the introduced DNA, that is, rearrangement of the transplastome has not occurred.

### Example 7

### Analysis of Transcription from Endogenous and Introduced psbA and rrn Promoters in Transplastomic Tobacco

Total RNA was isolated from well developed leaf tissue (Hughes and Galan 1988). Samples of 3 mg total RNA was separated in denaturing formaldehyde agarose gel (1.5%) and blotted to Hybond N+ membrane. The filters were UV crosslinked and then probed with ³²P labeled DNA. The same blot was used for hybridization using aadA, uidA psbA and 16S rRNA probes after stripping the probe each time.

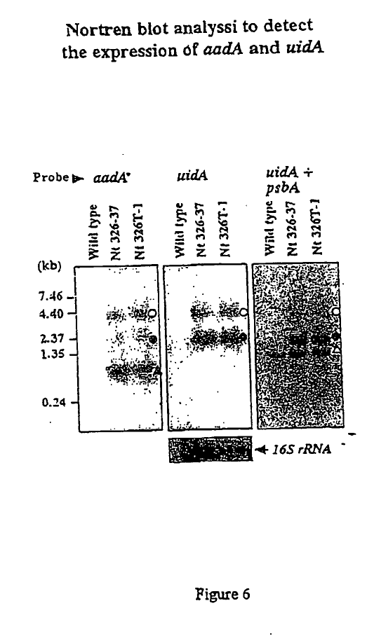

Northern blot analysis revealed high levels of expression of both rice rnn promoter driven aadA gene expression and rice psbA promoter driven uidA gene expression (FIG. 6). A 1.0 kb size transcript was observed in transplastomic lines when probed with aadA coding region.

Similarly, a 2.1 kb size uidA specific transcript was present in the total RNA of the transplastomic lines. Hybridization with uidA coding region also revealed the presence of a 4.0 kb dicitraonic rbcL-uidA message. The level of uidA mRNA was comparable with the level of native tobacco psbA mRNA. The steady state levels of psbA mRNA remained unchanged in all the transformed plants and the levels are comparable to wild type tobacco psbA mRNA levels indicating that the expression of uidA has, apparently, no deleterious effect on expression of native tobacco psbA gene. The aadA mRNA could not be compared to endogenously present 16S rRNA as the steady state levels of rRNA species are quite high due to its association with ribosomes. However, aadA mRNA levels were comparable with uidA mRNA levels.

### Example 8

### Analysis of Transcript Initiation from Rice Promoters in Tobacco Chloroplasts

Primer extensions were performed using Preamplification Superscript Kit (Gibco-BRL). The SR02 (SEQ ID NO: 2), SR14-16 (SEQ ID NO: 14-16) primers were labeled with (gamma ³²P) ATP and T4 polynucleotide kinase (Promega, USA). Ten micrograms of total RNA was used in each primer extension reaction. Size of the extension products was determined by comparison with DNA sequences generated with same primer using the Sequenase II kit (USB, USA).

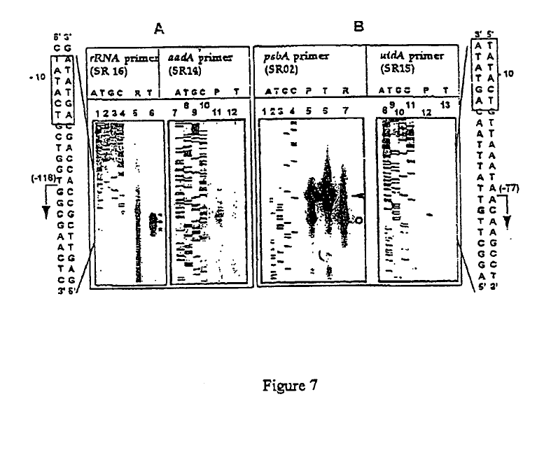

In order to establish that the chimeric genes were faithfully expressed from their own promoters and to locate the transcription initiation from the chimeric aadA and uidA genes, the 5′ end of transcripts was analyzed by primer extension studies (FIG. 7). The SR16 primer, homologous to rice (nucleotides 91,298-91,273 EMBL Acc. No. X15901) and tobacco (nucleotides 102,757-102,732, EMBL Acc. No. Z00044) rrn promoter was used to determine the transcription initiation site in tobacco, rice and one pVSR 326-transformed transplastomic line (Nt 326-37). The primer extension analysis showed that, in rice, 16S rRNA gene transcripts initiated from T (nucleotide 91,181 of rice chloroplast DNA, EMBL Acc. No. X15901) located 118 nucleotides upstream of mature 16S rRNA 5′ terminus (FIG. 7). In tobacco, three different transcripts, initiating from 116, 114 and 113 nucleotides upstream to mature 16S rRNA 5′ terminus, were observed using the same SR16 primer. Taking into account the 2 nucleotide difference in the 5′ unprocessed region between rice and tobacco, the migration of the primer extension product from rice corresponded to the −118 transcript of tobacco. Analysis of the Nt 326-37 transplastomic line using the aadA gene specific primer (SR14) revealed the presence of only one transcript corresponding exactly to the −118 transcript observed in wild type rice.

The SR02 primer, homologous to rice (nucleotides 1221-1200, EMBL Acc. No. X15901) and tobacco (nucleotides 1673-1652, EMBL Acc. No. Z00044) plastid DNA was used to determine the 5′ end of psbA transcript in tobacco, rice and Nt 326-37 line. The 5′ end of psbA gene transcript has been mapped 77 bases upstream to translation initiation ATG codon in rice and 85 bases upstream to ATG codon in tobacco. In the present study the 5′ end of the psbA transcript has been mapped using the SR02 primer for the tobacco and rice psbA promoter driven uidA genes. The rice psbA promoter is short by 8 nucleotides in the untranslated 5′ region between transcription initiation site (+1) and translation initiation codon (ATG) when compared to tobacco. In the primer extension reaction product of the Nt 326-37 transplastomic line, two different transcripts could be seen, one transcribed from native psbA gene of tobacco and the other transcribed from introduced chimeric uidA gene. This is because, where the SR02 primer anneals, the psbA promoter regions of rice and tobacco have a high degree of homology. The two transcripts differed exactly by 8 nucleotides. The primer extension analysis using the uidA gene specific primer (SR15) also mapped the same 5′ end for both the chimeric uidA transcript and the wild type rice pshA transcript.

### Example 9

### Analysis of GUS Protein Expression

Total soluble protein was extracted from well developed leaves of transgenic and control tobacco plants by homogenization in 50 mM Tris-HCl pH 7.0, 5 mM DTT, 1 mM Na₂EDTA, 0.1% SDS, 0.1% Triton X-100. The homogenate was centrifuged for 10 min in an Eppendorf centrifuge (4° C., 14,000 g) and protein concentration in the supernatant was determined by the. Bio-Rad Coomassie assay. For Western blot analysis, extracted proteins (10 mg per lane) were subjected to electrophoresis on 10% SDS polyacrylamide gels. Proteins were transferred by electroblotting to nitrocellulose membranes. The filters were blocked with phosphate buffer saline pH 7.2 (PBS) containing 0.5% dried milk. Blots were incubated with rabbit anti-b-glucoronidase serum (Clontech, USA) followed by goat anti-rabbit alkaline phosphatase (Sigma, USA). For quantification, GUS activity was assayed at 37° C. in GUS extraction buffer containing 1 mM 4-methyl umbelliferyl beta-D-glucuronide (MUG) as substrate. Fluorescence was measured on a Fluorometer Model TK0100 (Hoffer Scientific Instruments, USA) calibrated with 4-methylumbelliferone.

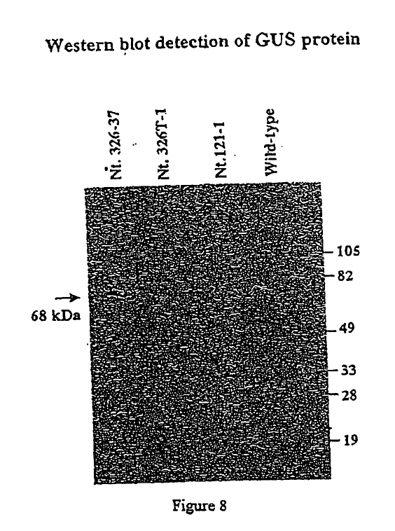

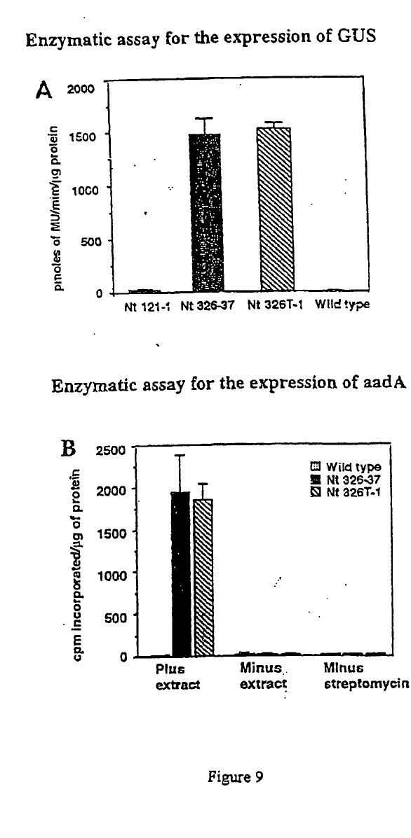

GUS protein expression was detected by Western blotting with anti-GUS antibodies (FIG. 8). Leaf extract from a plant nuclear transformed with the pBI 121 vector (Clontech), and containing single copy of uidA gene was also included in western blot analysis for comparison. No signal was observed in the Western blot when 10 mg of total soluble protein was loaded from a nuclear tansformed plant. On the other hand, a 68 kDa band corresponding to the expected size of GUS protein was present in the protein of transplastomic lines analysed. No signal was observed in the lane containing protein extracted from the control plant. A known amount of purified GUS protein (SIGMA, USA) was also included in the western blot analysis for quantification. The average GUS protein levels were similar for the six homotransplastomic lines studied and the highest level of GUS protein was found to be 2% of total leaf soluble protein. Enzymatic assay of GUS also confirmed the high level expression that was observed in the Western blot analysis. The activity of GUS in transplastomic plants was 80-100 fold higher when compared to the nuclear transformed plant using a spectrofluorometric method (FIG. 9, an average of 1500 pmole MU/min/mg of protein in transplastomic line viz 18 pmole MU/min/mg of protein in nuclear transformed plant).

### Example 10

### Analysis of aadA Expression

Expression of aadA was tested using the method described by Goldschmidt-Clermont 1991). The polypeptide encoded for by the aadA gene catalyses the transfer of an adenyl moiety from ATP to streptomycin. The assay is based on the binding of streptomycin adenylate but not ATP, to negatively charged phophocellulose paper (Whatmann P81). This activity could be detected in the plastid transformed plants. The level of expression was found to be the same in both Nt. 326-37 and Nt. 326T-1 plants.

### Example 11

### Progeny Analysis for Transplastome Stability, Maternal Inheritance and Uniform Expression of aadA and uidA Genes

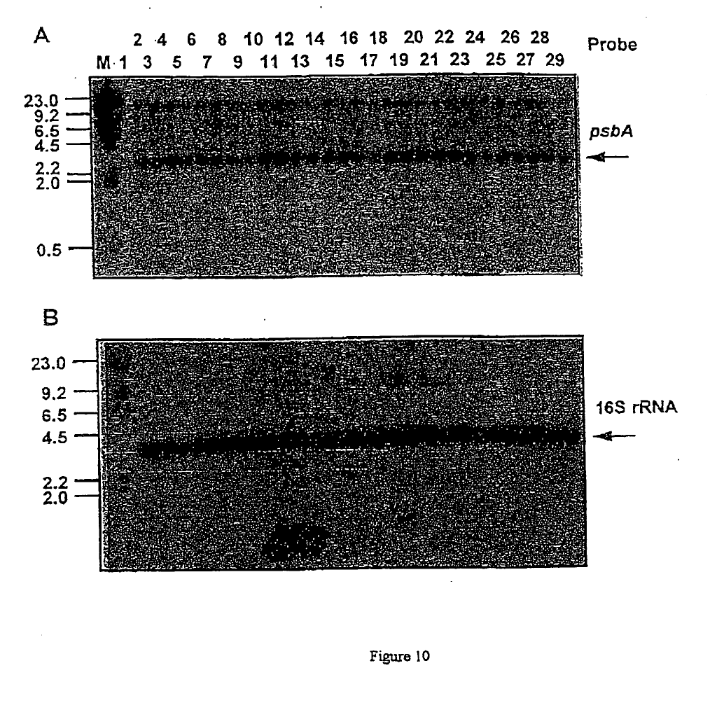

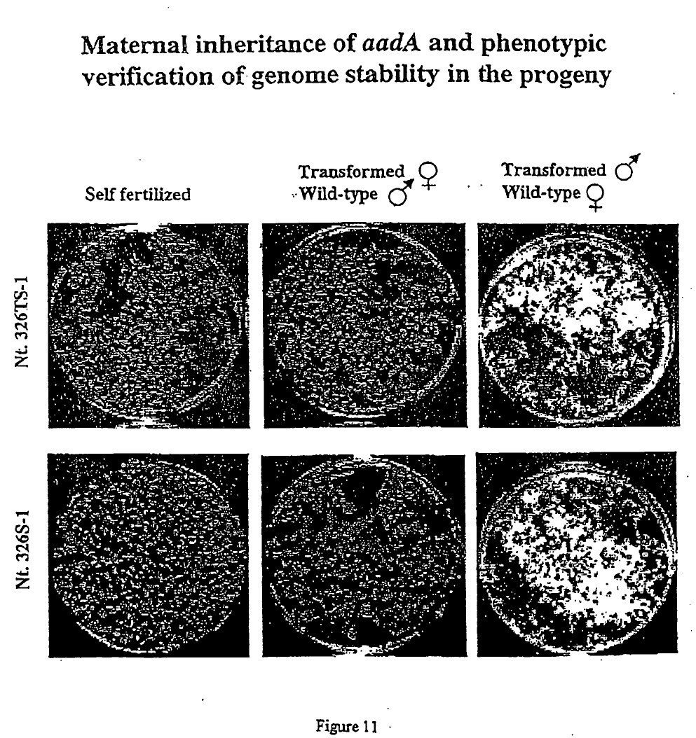

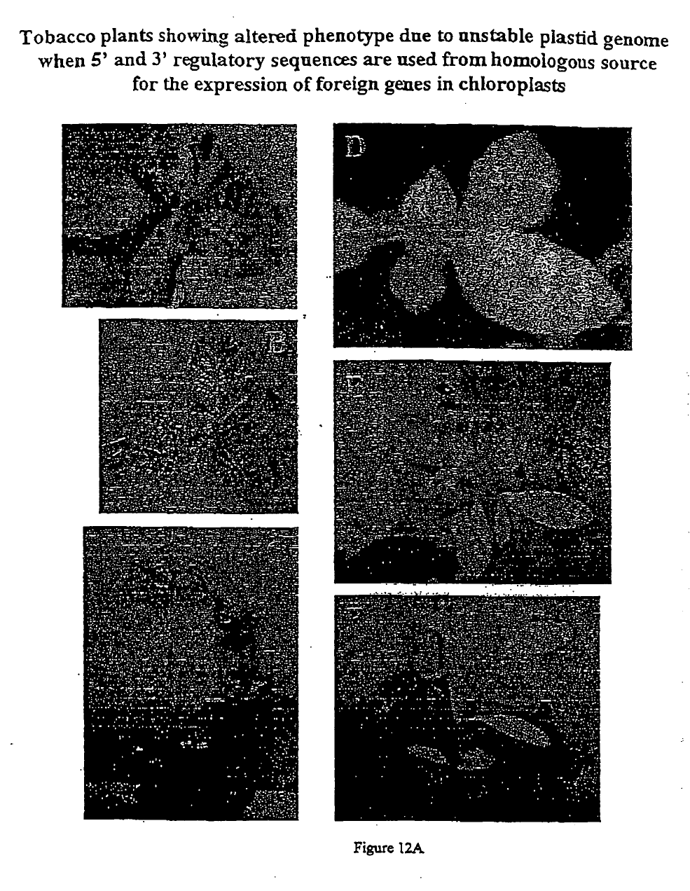

Progeny derived from the seed of selfed/reciprocal crosses of plants transformed by vector pVSR 326 were analyzed for the maternal inheritance and expression of antibiotic resistance by germinating the seedlings on 500 mg/L spectinomycin containing RM plates. The uidA expression was estimated by spectrofluorometer assay using MUG substrate. Total DNA isolated from the randomly selected individual seedlings was subjected to Southern hybridization analysis using pTB7, pTB19, psbA, rbcL-accD, 16S rRNA probes for transformed plastid DNA stability. To detect any possible rearrangements and/or deletions in the plastid DNA in the transformed plants, Southern hybridization analysis was carried out using various DNA probes and the results are presented in FIG. 10. In addition to nylon membranes used earlier for hybridization with aadA and uidA sequences, DNA isolated from single seedlings raised from twenty eight primary transformed plants digested with various restriction enzymes, blotted to nylon membranes were subjected to Southern hybridization using pTB7, pTB19, psbA, rbcL-aacD, 16S rRNA gene containing probes. 16S rRNA and rbcL-accD probes would detect the possible recombination between rice and tobacco rnn promoter regions and 3′ region of rbcL, respectively. Similarly, the probe containing psbA gene and its 5′ and 3′ regulatory regions would detect any recombination between rice and tobacco psbA regulatory regions. The identical pattern of hybridization that was observed among all the transplastomic lanes using all the probes confirmed the stable inheritance of transgenes into the progeny. These results clearly indicate that the aadA and uidA genes are integrated only in the site specified by the flanking region (rbcL-accD locus) through homologous recombination without any rearrangements in the plastid DNA due to further recombination between regulatory regions. Maternal inheritance of aadA and uidA was verified by testing the expression of GUS activity and phenotypically verifiable antibiotic resistance in the selfed seed progeny and from reciprocal crosses (FIG. 11). The aadA expressing seedlings remain green and the non expressing seedlings will turn white when germinated on spectinomycin containing plates. The seedlings raised from the seed of selfed primary transformants and from the reciprocal crosses involving transformed plant as female parent remained green indicating the maternal inheritance of antibiotic resistance. Uniform GUS activity was observed among all the progeny plants obtained from the selfed seed.

### Example 12

### Expression Vectors for ifnG in Chloroplasts

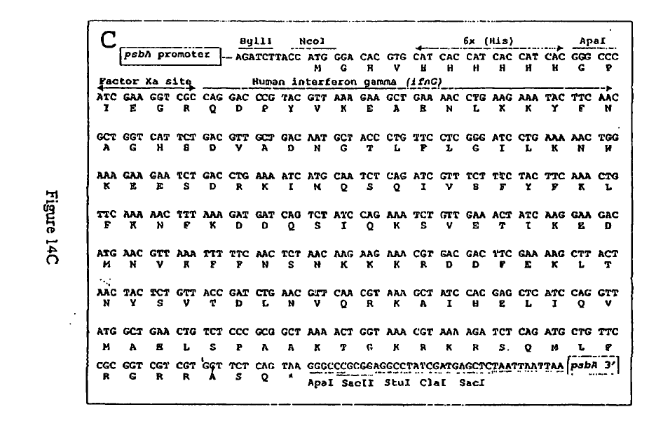

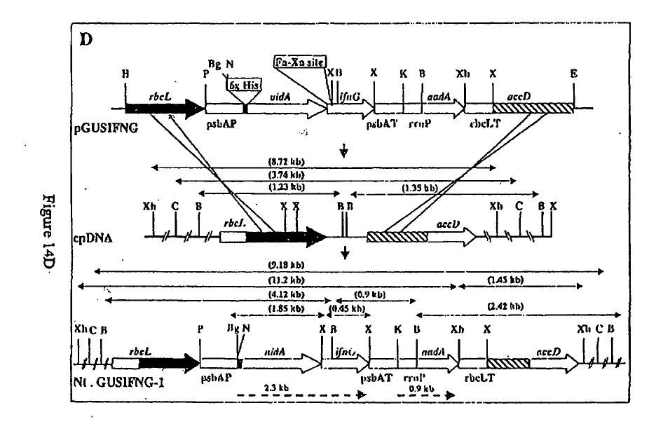

The p326IFNG was a derivative of vector pVSR326. In the pVSR326, the uidA was replaced with a multiple cloning site through the insertion of SR45 (SEQ ID NO: 45) and SR46 (SEQ ID NO: 46) primers that were complimentary to each other at the BgIII and SacI sites to create pVSRIFNG1. In the next step, (6×) His-tag was introduced using SR47 (SEQ ID NO: 47) and SR48 (SEQ ID NO: 48) primers that were partially complimentary to each other at NcoI and ApaI sites to create pVSRIFNG2. The ifnG coding region (FIG. 14C) was PCR amplified from an E. coli expression vector pPLIFNG (Wang et al, 1992) using SR49 (SEQ ID NO: 49) and SR50 (SEQ ID NO: 50) primers and cloned at ApaI site of pVSRIFNG2 to create p326IFNG.

For the construction of pGUSIFNG, the uidA coding region, devoid of stop codon, was PCR amplified from pGUSN358→S (Farrell and Beachy 1990) using SR51 (SEQ ID NO: 51) and SR52 (SEQ ID NO: 52) primers and cloned into vector pQE30 (Qiagen) at BamHI and KpnI sites to create pQEGUS. Parallely, the ifnG coding region was PCR amplified from pPLIFNG using SR53 (SEQ ID NO: 53) and SR54 (SEQ ID NO: 54) primers and cloned into vector pQE31 (Qiagen) at the PstI site to create pQE31IFNG. The uidA along with T7 promoter and (6×) His-tag was released from pQEGUS as BamHI and SmaI fragment and cloned into pQE31 IFNG digested with KpnI (end filled) and BamHI to create vector pQEGUSIFNG. Finally, the uidA:ifnG fusion gene was PCR amplified from pQEGUSIFNG using SR47 and SR55 (SEQ ID NO: 55), digested with NcoI and cloned into NcoI and SacI (end filled) digested pVSR326 to create vector pGUSIFNG.

The vector pBIIFNG was created by cloning PCR amplified ifnG from pPLIFNG using SR56 (SEQ ID NO: 56) and SR57 (SEQ ID NO: 57) primers into vector pBI121 (Clonetech) at XbaI site.

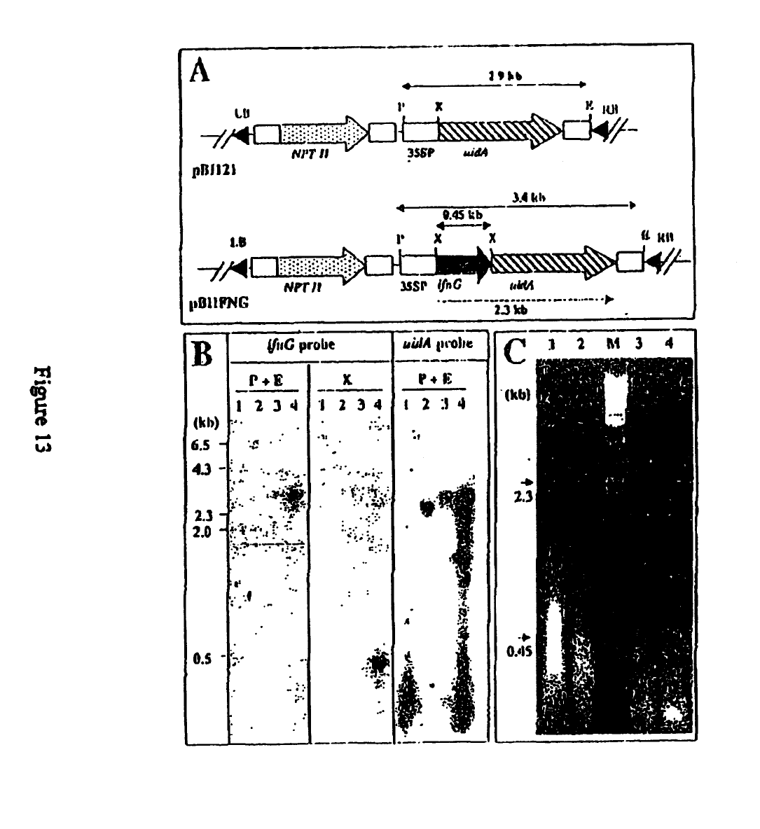

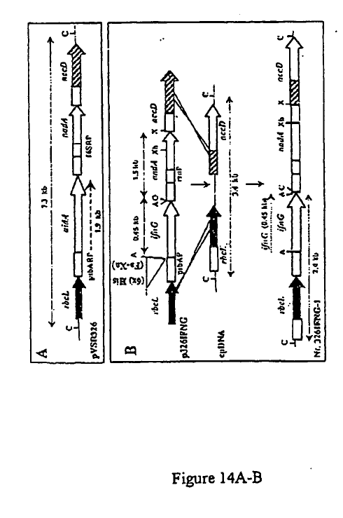

To produce and purify recombinant human IFN-g, the ifnG was transformed into tobacco nucleus or plastid genome to express it as an individual or as a fusion protein. A binary vector pBIIFNG derived from pBI121 was used for the nuclear expression (FIG. 13A). In the pBIIFNG, the coding region of ifnG was transcriptionally fused to a reporter uidA gene (GUS) in pBI121 vector. Both uidA and ifnG have their own translation initiation (ATG) and termination (TGA) codons and both the genes were under the transcription control of the same CaMV 35S promoter. Two vectors, p326IFNG and pGUSIFNG, were used to express IFN-g in tobacco chloroplasts as an individual and as a fuision protein, respectively. Both the vectors were derived from pVSR326 that contained a selectable aadA gene that confers resistance to spectinomycin and a reporter uidA gene under the regulation of rice psbA and rrn promoters, respectively (FIG. 14A). The rbcL-accD gene sequences derived from tobacco plastid genome were provided in the vector flanking the transgenes for site-specific integration through two homologous recombinations. The p326IFNG was obtained from pVSR326 by replacing uidA with that of ifnG (FIG. 14B). The complete nucleotide and the deduced amino acid sequences along with features incorporated to express and purify the IFN-g were presented in FIG. 14C. A (6×) His-tag was added in frame at the N-terminal end of IFN-g to purify recombinant protein using an Ni-NTA column. A protease site, IEGR, recognized by factor Xa was introduced in-between the His-tag and IFN-g to allow cleavage of the His-tag from the recombinant IFN-g after purification. In the vector, PGUSIFNG, the ifnG was translationally fused at the C-terminal end of uidA (FIG. 14D). The (6×) His-tag was added at the N-terminal end of GUS:IFN-g and a factor Xa recognition site was introduced at the fusion junction (FIG. 14D). The IFN-g released upon the cleavage of fusion protein by factor Xa will thus contain the same amino acid sequence as that of mature IFN-g produced in the human body. The direction and size of transcripts from uidA, ifnG, uidA:ifnG and aadA genes, a possible mechanism for transgene integration into the tobacco plastome and the size of DNA fragments from restriction digestion with relevant enzymes is shown in FIG. 14A-D.

### Example 13

### Transformation and Regeneration of Stable Transgenic Plants

The Agrobacterium mediated transformation method was followed for nuclear transformation of tobacco with pBI121 and pBIIFNG binary vectors under kanamycin selection. Particle bombardment of leaf tissue was used for chloroplast transformation under spectinomycin selection using DNA of vectors pVSR326, p326IFNG and pGUSIFNG. Tobacco (Nicotiana tabacum cv. Petit Havana) was transformed using particle delivery system PDS1000 (BioRad) according to the method described by (Svab and Maliga 1993). In brief, vector DNA coated on to tungsten particles (M17 Bio-Rad) was bombarded on the abaxial side of a tobacco leaf placed on RMOP medium (Svab and Maliga 1993), a modified MS medium (Murashige and Skoog 1962) containing 0.1 mg/l thiamine, 100 mg/l inositol, 3% sucrose, 1 mg/l BA and 0.1 mg/l NAA, 0.6% agar, pH 5.8). Transformed shoots were selected on RMOP medium containing 500 mg/l spectinomycin dihydrochloride.

Three additional cycles of regeneration on spectinomycin (500 mg/l) containing RMOP medium was carried out to obtain homotransplastomic plastid containing plants (Svab and Maliga 1993).The Agrobacterium strain LBA 4404 containing vector pBIIFNG/pBI121 was used for nuclear transformation following a leaf disc method (Horsch 1985).

Although the vector DNA is randomly delivered into leaf cells in the particle bombardment method, the selectable aadA is expected to express and confer resistance to spectinomycin only when it entered the chloroplasts due to the specificity of the rrn promoter. Substantially, homotransplastomic lines were established by repeating regeneration process three times from the leaf tissues of primary transformants under spectinomycin selection. Unless and otherwise mentioned, Nt. BI121-1, Nt. BIIFNG-½, Nt. VSR326-37, Nt. 326IFNG-½ and Nt. GUSIFNG-1 plants transformed with vectors pBI121, pBIIFNG, pVSR326, p326IFNG and pGUSIFNG, respectively, were subjected to molecular analysis to confirm the transgenic nature of regenerated plants and the expression of recombinant IFN-g protein. Total DNA isolated from transgenic and control plants (Mettler 1987) was digested with relevant restriction endonucleases, separated on 0.8% agarose gels and transferred on to nylon membrane. About 3 μg of total RNA isolated from leaf tissue (Hughes and Galam 1988) was separated in denaturing formaldehyde agarose gel (1.5%) and blotted to nylon membranes. The membranes were UV crosslinked and then probed with ³²P labeled psbA (SEQ ID NO: 39), 16S rRNA (SEQ ID NO; 32), uidA (SEQ ID NO: 33), aadA (SEQ ID NO: 34) and targeting sequence (SEQ ID NO: 35) amplified using SR17 (SEQ ID NO: 17)—SR18 (SEQ ID NO: 18), SR19 (SEQ ID NO: 19)—SR20 (SEQ ID NO: 20), SR41 (SEQ ID NO: 41)—SR42 (SEQ ID NO: 42), SR43 (SEQ ID NO: 43)—SR44 (SEQ ID NO: 44) and SR12 (SEQ ID NO: 12)—SR13 (SEQ ID NO: 13) primer pairs, respectively. Standard procedures were followed for hybridization (Sambrook et al, 1989) and membranes were subjected to autoradiography.

### Example 14

### Integration of uidA, aadA, ifnG into Nuclear/plastid Genomes

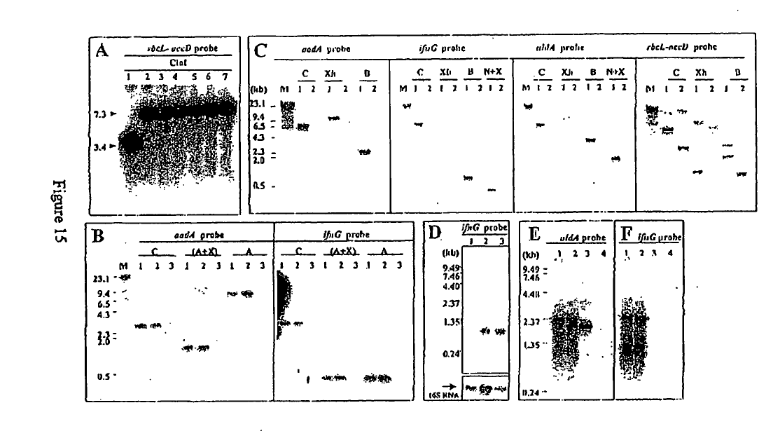

Southern hybridization analysis using uidA, aadA, ifnG and rbcL-accD probes confirmed the stable integration of vector DNA into tobacco nuclear and plastid genomes (FIG. 13B, FIG. 15A-C). The genomic DNA isolated from Nt. BIIFNG-1, Nt. BIIFNG-2 and Nt. BI121-1 plants were subjected to Southern hybridization analysis. The presence of 2.65 kb and 0.45 kb size fragments can be seen in the PstI-EcoRI and XbaI digested DNA, respectively, when probed with ifnG coding regions (FIG. 13B). Reprobing the blot containing PstI-EcoRI digested DNA with uidA reconfirmed the presence of an expected 2.65 kb fragment (FIG. 13B).

Integration of vector pVSR326 into the plastid genome was confirmed by digesting the total genomic DNA with ClaI and probing with uidA and aadA gene probes. Presence of a 3.4 kb signal in the wild-type plant (FIG. 15A, lane 1) and a 7.3 kb signal in all six transformed plants tested (FIG. 15A, lanes 2-7) when probed with the rbcL-accD sequences confirmed the site-specific integration of uidA and aadA. The complete absence of a 3.4 kb signal in transplastomic lines is a clear evidence for the homoplasmic nature of the transplastome. Similar analysis was performed to confirm the transgenic nature of Nt. 326IFNG-1, Nt. 3261FNG-2 (FIG. 15B) and Nt. GUSIFNG-1 (FIG. 15C). As shown in FIG. 15B, the size of the fragments that hybridized to the aadA and ifnG in Nt. 3261FNG-1 and Nt. 326IFNG-2 plants were in agreement with the predicted size DNA fragments when transgenes are integrated into the plastid genome site-specifically. Similarly, Southern hybridization confirmed the stable integration of uidA:ifnG into Nt. GUSIFNG-1 plastome (FIG. 15C). As predicted by the restriction map, all the three probes (aadA, ifnG and uidA) hybridized to a common 11.2 kb and 9.18 kb band in XhoI and ClaI digested DNA, respectively (FIG. 15C). Hybridization with the targeting rbcL-accD probe further confirmed the stable site-specific integration of uidA:ifnG and aadA into the plastome (FIG. 15C). The complete absence of 3.74 kb and 8.72 kb signals in ClaI and XhoI digested DNA in the rbcL-accD hybridized blot is a clear evidence for the complete homoplasmic nature of the transplastome in Nt. GUSIFNG-1 plant

### Example 15

### Transcription of Chimeric uidA, ifnG and uidA:ifnG Genes

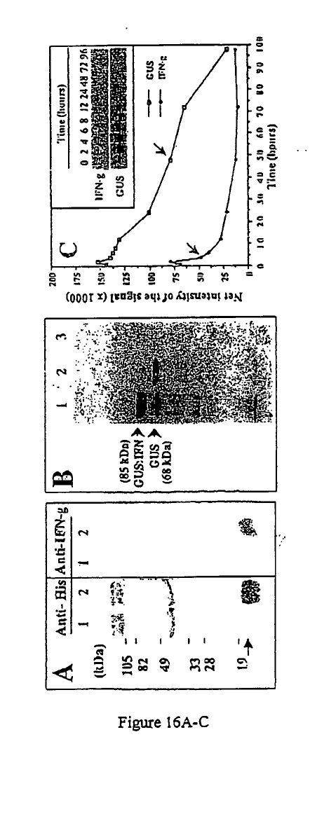

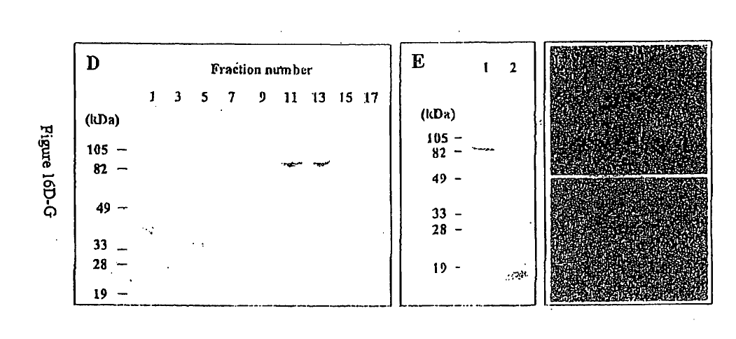

In the Nt. BIIFNG-1 plant, transcription of ifnG gene was examined by Northern hybridization (data not shown) and RT-PCR (FIG. 13C). A 2.3 kb size DNA fragment amplified in RT-PCR (FIG. 13C, lane 2) when ifnG (SR56) and uidA (SR18) gene specific primers were used, confirmed the presence of a fusion transcript in the RNA from Nt. BIIFNG-1. No amplification was observed when cDNA made from RNAse treated RNA was used as a template (FIG. 13C, lane 1). The RT-PCR analysis using ifnG gene specific primers (SR56 and SR57) amplified a fragment of about 0.45 kb in size from cDNA template made from DNAse treated RNA. In the chloroplast transformed plants, Northern blot analysis was performed to confirm the transcription of chimeric uidA, aadA, ifnG and uidA:ifnG genes (FIG. 15D-F). As shown in FIG. 15D, a 0.45 kb transcript corresponding to the expected size of ifnG was observed in the RNA from Nt. 326IFNG-1 and Nt. 3261FNG-2 plants. Hybridization using uidA probe confirmed transcription of uidA and uidA:ifnG in Nt. 326-37 and Nt. GUS:IFNG-1 plants, respectively (FIG. 15E and F). The uidA transcript levels in Nt. 326-37 were comparable with the uidA:ifnG transcript levels in Nt. GUS:IFNG-1, indicating that the fusion of ifnG to uidA had no adverse effect on fusion gene transcription. Reprobing the same blot with ifnG reconfirmed the presence of 2.3 kb fusion transcript in the Nt. GUSIFNG-1 (FIG. 15F, lanes 1 and 2). The RNA sample from Nt. VSR326-37 (FIG. 15E, lane 3) included in the blot for direct comparison of uidA and uidA ifnG transcript levels additionally confirmed the presence of expected size uidA:ifnG fusion transcript in Nt. GUSIFNG-1 plant (FIG. 16E, lanes 1 and 2).

### Example 16

### Expression of GUS, IFN-g and GUS:IFN-g

For Western blotting (Sambrook et al, 1989), proteins at various stages of purification (see below) were subjected to SDS-PAGE (Laemmli 1970), electroblotted on to nitrocellulose membranes, blocked with bovine serum albumin (BSA), incubated by anti-GUS (Clontech), anti-IFN-g (Calbiochem), anti-His antibodies (Qiagen) and detected by goat anti-rabbit alkaline phosphatase (Sigma) as per the supplier instructions.

For pulse labeling, total protein was labeled by incubating the leaf discs in 10 ml of MS medium containing 3.5 mCi labeled amino acid mix (S-35 Express, NEN/DuPont) and incubated at 25° C. under 4,000 lux light. After one hour, leaf discs were thoroughly washed with MS medium and continued the incubation for 96 hours. At various defined intervals, leaf discs were quickly frozen in liquidA nitrogen and protein extracted, immunoprecipitated (Pineiro et al 1999) with anti-His antibodies (Qiagen), separated on SDS-PAGE, transferred onto nitrocellulose membrane and subjected to autoradiography. The signal intensity was quantified using ID Image analysis software (Kodak).

Western blotting was used to confirm the presence of GUS, IFN-g and GUS:IFN-g proteins in Nt.326-37, Nt., 3261FNG-1 and Nt. GUSIFNG-1 plants, respectively. In addition to anti-IFN-g, the anti-His antibodies were also expected to recognize the recombinant IFN-g produced in the Nt. 3261FNG-1 plant, as it contained a (6×) His-tag at the N-terminal region. As shown in FIG. 16A, both anti-His and anti-IFN-g antibodies recognized a 17 kDa protein, an expected size for IFN-g, in Nt. 326IFNG-1, confirming the expression of IFN-g. Similar analysis using anti-His-tag, anti-IFN-g (data not shown) and anti-GUS antibodies revealed the presence of a 85 kDa GUS:IFN-g fusion protein in the leaf extracts of Nt. GUSIFNG-1 (FIG. 16B). The estimated levels of IFN-g and GUS was found to be 0.01% and 3% of the total cellular protein in the leaf extracts of Nt. 326IFNG-1 and Nt. 326-37 plants, respectively. To understand the reasons for 300 fold difference in the expression levels between IFN-g and GUS, the role of protein degradation was investigated. The leaf discs from Nt. VSR326-37 and Nt. 326IFNG-1 plants were pulse labeled with S-35 (Methionine and Cysteine) to analyze the half life of recombinant IFN-g and GUS proteins in the chloroplasts. As shown FIG. 16C, the GUS protein had a half life of about 48 hours whereas IFN-g had comparatively very short half life (about 4-6 hours) in chloroplasts.

### Example 17

### Purification of IFN-g and Assaying its Biological Activity

Soluble leaf protein extract obtained from the greenhouse grown Nt. GUS:IFN-g-1 plant in buffer A (50 mM Tris-HCl pH 7.0, 5 mM DTT, 1 mM Na2EDTA, 0.1% SDS, 0.1% Triton X-100, one protease inhibitor cocktail tablet per each 50 ml of buffer) was loaded onto a 200 ml DEAE cellulose column equilibrated with the same buffer. The column was washed with 5 vol buffer B (buffer A, 50 mM NaCl) and the bound proteins were eluted with 50-500 mM NaCl gradient in buffer A. The GUS positive fractions, eluted between 100-200 mM NaCl were pooled and directly loaded on to a 15 ml Ni-NTA agarose column. The column was washed with 4 vol of buffer C (100 mM potassium phosphate buffer pH 8, 20 mM imidazole) and eluted with a 20-250 mM imidazole gradient in buffer C. The fractions with peak GUS activity were dialyzed against buffer D and fusion protein was cleaved (in 4 mg batches) by the incubation with factor Xa. The biotinilated factor Xa cleavage and removal kit (Boeringer Mannheim) was used to separate the IFN-g from the fusion protein as per the supplier instructions. After the cleavage, protein was passed once again through a fresh Ni-NTA column to remove the undigested GUS:IFN-g and GUS protein containing His-tag. The flow through was loaded directly on to a 10 ml S-sepharose column equilibrated with buffer E (20 mM Tris. Hcl pH 8). After washing the column with 50 ml buffer E, the bound protein was eluted with 80 ml buffer E+1.2 M NaCl gradient. The fractions containing recombinant IFN-g was dialyzed extensively, analyzed on SDS-PAGE for purity, quantified the protein and then subjected to bioassay (Lewis 1988). In brief, the human lung carcinoma cells precultured for 24 h in the presence or absence of rh-IFN-g were challenged with 104 PFU of encephalomyocarditis (EMC) virus. One unit of antiviral activity was defined as the amount of rh-IFN-g required to produce equivalent antiviral activity expressed by 1 U of the (NIH IFN-G reference standard (Gg 23-901-530)).

Only 10% of the total estimated GUS:IFN-g fusion protein with less than 85% purity, as judged on a comassie blue stained SDS-PAGE gel, was recovered when purified directly using Ni-NTA column. However, the fusion protein was purified to near homogeneity using a two step column procedure (Table 1).

TABLE 1
Purification of GUS:IFNG fusion protein starting from 50 grams (fresh
weight) of greenhouse grown plant leaves

| | Total protein | Total | Specific activity | Yield |
|---|---|---|---|---|
| Purification step | (mg) | (U) | (U/mg) | (%) |
| Crude extract | 750 | 5,791,666 | 7,722 | 100 |
| Cellulose DE52 | 353 | 4,866,000 | 13,784 | 84 |
| Ni-NTA colum | 18 | 4,402,000 | 244,555 | 76 |

(One unit is defined as the amount of activity required to release one μmole of MU from MUG in one minute at 37° C.)

In the first step, the crude extract was loaded on to DE-52 column and the bound proteins were eluted with 0-1.0 M salt gradient. These fractions were tested for the presence of GUS:IFN-g fusion protein by a GUS assay. The colorless X-GlcU substrate containing solution turned to blue color in less than 5 min when the fractions that contained highest amounts of GUS:IFN-g fusion protein were assayed.

The activity of GUS was detected by histochemical assay and measured by fluorometric assay (Gallagher 1992). The fractions containing GUS:IFN-g fusion protein were identified using a histochemical X-GlcU substrate. The colorless X-GlcU substrate produced a visually detectable blue indigo dye upon the addition of a small aliquot of fractions that contained GUS:IFN-g protein when incubated at 37° C. Protein concentration was determined with the Bradford reagent (BioRad) using BSA as standard. The antiviral activity of recombinant IFN-g was assayed following the standard procedures (Lewis 1988) using human lung carcinoma (A 549) cells and encephalomyocarditis (EMC) virus. The expression levels of IFN-g and GUS were quantified by comparing with E. coli derived IFN-g (Boeringer Mannheim) and GUS protein (Sigma) standards, respectively, using ELISA method.

The GUS-positive factions were loaded onto an Ni-NTA column directly and the bound protein was eluted with 0-250 mM imidazole gradient. All the fractions were subjected to GUS assay and analyzed on SDS-PAGE (FIG. 16D). Using this procedure, we were able to obtain about 18 mg of fusion protein (75% recovery) starting from 50 grams of fresh leaf material. The IFN-g was separated from the GUS using a biotinilated factor Xa cleavage and purification procedure. After the cleavage, the biotinilated factor Xa was removed using strepatavidin beads and the protein was passed through a second Ni-NTA column to remove the His-tag containing GUS protein. Finally, the IFN-g present in the flow through from the second NI-NTA column was purified on an S-sepharose column with an estimated recovery of 70% protein. The recombinant IFN-g was found to be highly pure, as judged by commassie blue staining (data not shown), and cross reacted with anti-IFN-g antibodies (FIG. 16E, lane 2). In a bioassay, a 24 h pretreatment of human lung carcinomas (A 549) with 25 pg of the purified IFN-g offered complete protection against the infection of by EMC virus (FIG. 16F), whereas the untreated cells were completely infected (FIG. 16G). These results suggested that the purified recombinant IFN-g have a specific activity of>10⁷ IU mg⁻¹ protein.

### Discussion of Examples 12-17

The expression of IFN-g was very low (0.001%) in the nuclear transformed plants despite of the fact that the ifnG was placed under the regulation of a strong and constitutive CaMV 35S promoter. Low expression of foreign genes is not uncommon in transgenic plants (Goddijin and Pen 1995). Although it is difficult to compare the expression levels of various proteins expressed in the nuclear transformed plants due to variations in the promoters used, copy number of integrated gene, site of integration and methods followed for protein extraction, the expression levels are generally low, especially, when compared to microbial expression systems (Goddijin and Pen 1995). Considerable improvement in the expression level was achieved through the modification of codon usage (Perlok et al, 1991), by the addition of ER retention signals (Fiedler et al, 1997), by subcellular targeting (Wong et al, 1992; Fiedler 1997; Schouten et al, 1996; 1997) and through the use of strong promoters in combination with enhancer elements (Fiedler et al, 1997). Recently, very high level expression of maize phosphoenolpyruvate carboxylase was reported when transformed into rice plants accumulating up to 12% of the total leaf soluble protein (Ku et al, 1999). However, the same gene expressed at low level in tobacco (Hudspeth et al, 1991) and potato (Gehlien et al, 1996) suggesting that such high level expression is specific for few taxonomically related species (Ku et al, 1999).

So far, the expression levels of foreign genes in transplastomic plants were found to be very high due to possible amplification of gene copy number. Amplification of Bacillus thuringiensis cryIA(c) gene in chloroplasts led to extraordinarily high levels, 3-5% of the soluble protein, of insecticidal protein production (McBride et al, 1995). Recently, expression of aroA from petunia that confer low levels of tolerance to glyphosate, a herbicide, was shown to confer very high levels of tolerance due to overproduction of enzyme 5-enol-pyruvyl shikimate-3-phosphate synthase (EPSPS) when expressed in chloroplasts (Daniell et al, 1998). Similarly, expression of uidA resulted into high level accumulation of GUS in chloroplasts reaching up to 2.5% of the total cellular protein (Stuab and Maliga 1993). In the present study, similar level of GUS expression (3%) was observed in the chloroplast transformed Nt. 326-37 plant using vector pVSR326.

In order to improve the IFN-g expression, the ifnG was cloned into plastid transformation vector p326IFNG and transformed into tobacco chloroplasts. Although there was 100 fold increase in the IFN-g expression levels in the plastid transformed Nt. 326IFNG-1 plant when compared to nuclear transformed Nt. BIIFNG-1 plant, these expression levels are 200-300 fold low when compared to GUS expressed under the same psbA promoter and integrated into the plastid genome at the same site. One of the reasons for such low levels of IFN-g expression could be due to the lack of efficient transcription/mRNA stability and/or fast degradation of the recombinant protein. The Northern hybridization analysis revealed efficient transcription of ifnG in Nt. 326IFNG-1 plant at a level that is comparable with that of uidA transcription in Nt. 326-37 plant expressed under the same promoter, ruling out the possibility of low levels of transcription or mRNA stability as a cause for low ifnG expression. On the other hand, the pulse labeling experiments have shown that the IFN-g has relatively a short half life (4-6 hours) in chloroplasts as compared to 48 hours for GUS suggesting that the rapid degradation of IFN-g was the reason for such a low accumulation.

To increase the expression levels of IFN-g further, the ifnG was translationally fused to high expressing uidA in chloroplasts and integrated again into tobacco chloroplast genome. In the Nt. GUSIFNG-1 plants, about 5% of the total cellular protein were found to be of GUS:IFN-g fusion protein. When compared to 0.001% expression of IFN-g in the nuclear transformed plants and 0.01% in the chloroplast transformed plants, the 5.0% expression levels achieved through GUS-fusion strategy are exceptionally significant. Therefore, GUS fusion offers an attractive way to increase the low expressing proteins/peptides in transgenic plants. Recently, enhanced expression of antimicrobial peptide sarcotoxin IA was reported by GUS fusion in transgenic tobacco plants (Okamoto et al, 1998). One key advantage of GUS fusion system is it's ability to accept N- and C-terminal fusions without any loss of it's activity (Jefferson et al, 1989). This ability of GUS has been widely exploited for detecting sub-cellularly targeted proteins and in tracking the virus movement in plants. In the present study, we have exploited this property during the protein purification to identify fractions containing GUS:IFN-g fusion protein very rapidly using a simple and inexpensive GUS assay (Gallagher 1992). This feature is particularly useful for those proteins that are highly labile under certain harsh conditions employed during purification steps and difficult to detect with simple analytical assays. Therefore, the GUS fusion system combined with chloroplast transformation described here offers key advantages in increasing the yields of poorly accumulating proteins and reduce purification time considerably. It should be mentioned here that, so far, GUS is the safest and most commonly used reporter in transgenic plants (Gilissen et al, 1998).

The number of steps involved in the downstream processing to recover the recombinant protein from relatively large volumes of plant biomass play critical role in the use of plants as bioreacters (Krebbers and Vandekerckhove 1990, Goddijin and Pen 1995). Affinity based purification greatly reduces the number of chromatography steps involved in the purification of protein. Translational fusion of (6×) His-tag at the N- or C-terminal end facilitates separation of recombinant protein using nikel-nitrilotriacetic acid (Ni-NTA). While such a strategy has been often used for the purification of E. coli expressed proteins (Hochuli et al, 1987), it has been rarely used in plant systems (Flachmann and Kuhlbrandt 1996; Sugiura 1999). The His-tag can be removed, if necessary, by providing a specific protease site separating His-tag from the recombinant protein. Unlike E. coil expressed proteins, our efforts to purify GUS:IFN-g fusion protein from Nt. GUSIFNG-1 plant leaf extracts in a single step using Ni-NTA column was futile and yielded only 10% of the total estimated protein. This might be due to the presence of a vast pool of free histidine and other substances in the crude extract that might be competing directly for Ni-NTA binding. However, the GUS:IFN-g fusion protein was purified to near homogeneity by a two column purification process involving DE 52 followed by Ni-NTA column. Biotinilated factor Xa cleavage and removal procedure was adapted to separate IFN-g from the GUS fusion partner and the cleaved-IFN-g was purified further by S-sepharose column. The purified recombinant human IFN-g offered complete protection against the infection of human lung carcinomas (A 549) by EMC virus suggesting that IFN-g expressed in chloroplasts folded correctly and retained it's biological activity at a level that is comparable to E. coli derived r-IFN-g. In the present study, as a test case, we have used His-tag. However, there are number of ligands described in the literature for the purification of recombinant proteins expressed in microbial, yeast, insect and mammalian systems which can replace His-tag depending on the need.

During the past one decade, a number of laboratories have experimented with the use of plants for “biomanufacturing” of specialty products as plants can express, fold, assemble and process foreign proteins (Krebbers and Vandekerckhove 1990; Goddijin and Pen 1995). The recent clinical trials in humans of plant expressed heat labile enterotoxin subunit B (LT-B) from enterotoxogenic E. coli for edible vaccine (Tacket et al, 1998) and a monoclonal secretary antibody that recognize surface adhesion protein of Streptococcus mutans as a preventive immunotherapy (Ma et al, 1998) have further strengthened this approach. Plant derived proteins with high level of purity may be more readily acceptable than the similar products obtained from bacteria and transgenic animals due to possible contamination by human pathogens (Miele 1997). There are a number of small proteins/peptides that are highly useful in the pharmaceutical industry if made available in bulk and at a low-cost (Ellis 1996; Goddijin and Pen 1995; Krebbers and Vandekerckhove 1990; Rudolph 1999). Initially, it may be easy to couple such production systems with the existing and well organized floriculture industry (Miele 1997). Floriculture, a multi-billion dollar industry spreading all over the world, utilizes weather controlled greenhouses for the production of quality cut flowers. The greenhouse grown plants being relatively free from pests and pathogens when compared to the open field cultivated plants may offer certain advantages and qualify better in case of stringent quality control tests imposed by various national/international health agencies/organizations (Miele 1997). In floriculture, only a fraction of the plant biomass (flowers) is harvested and the majority of biomass consisting of mostly leaf material go waste. The extension of chloroplast transformation in conjunction with fusion protein and affinity based purification strategies to floricultural crops can result in the addition of substantial value to the crop without any additional inputs and convert the so far unutilized plant biomass as a raw material for new industrial applications benefitting both farmer and industry immensely while providing better health for mankind.

### References

Altschul et al (1990) J. Mol. Biol. 215: 403-410

Altschul (1993) J. Mol. Evol. 36: 290-300

Arora & Khanna (1996) J. Biotechnol. 52: 127-133

Bemiller, et al (1995) Blood Cells Mol. Dis. 21: 23947

Blowers et al (1989) Plant Cell 1: 123-132

Blowers et al (1990) Plant Cell 2: 1059-1070

Boynton et al (1988) Science 240: 1534-1538

Cannon, et al (1990) J. Rheumatol. 17: 304-10

Chong, et al (1997) Transgenic Res. 6: 289-296

Chong, et al (1997) Gene 192: 271-281

Devereux et al (1984)Nucl. Acids. Res. 12: 387-395

Daniell, et al (1998) Nat. Biotechnol. 16: 345-348

Daniell (1999) Nature Biotech. 17: 855

di Guan, et al (1988) Gene 67: 21-30

Dobrovolsky, et al (1993) FEBS lett 319: 181-184

Ealick, et al (1991) Science 252: 698-702

Ellis, et al (1999) Clin. Immunol. 92: 49-55

Ellis, et al (1996) Current Opinion in Biotechnology 7: 646-652

Farrell, et al (1990) Plant Mol. Biol. 15: 821-825

Fiedler, et al (1997) Immunotechnology 3: 205-16

Flachmann, et al (1996) Proc. Natl. Acad. Sci. 93: 14966-14971

Gallagher, et al (1992) Acadamic Press, Inc. New York

Gehien, et al (1996) Plant Mo l. Biol. 32: 831-848

Gilissen, et al (1998) Transgenic Res. 7: 157-63

Goddijin, et al (1995) TIBTECH. 13: 379-387

Golds et al (1993) Bio/Technology 11: 95-97

Goldschmidt-Clermont (1991) Nucl. Acids. Res. 19: 4083-4089

Gray, et al (1982) Nature 295: 503-508

Grierson & Covey (1988) Plant Mol. Biol. (2^{nd} Edition). Chapman and Hall, New York, 64-65

Hanifin, et al (1993) J. Am. Acad. Dermatol. 28: 189-197

Henikoff& Henikoff(1992) Proc. Natl. Acad. Sci. USA 89: 10915-10919

Hiratsuka et al (1988) Mol. Gen. Genet 217: 185-194

Hochuli, et al (1987) J. Chromatography 411: 177-184

Horsch, et al (1985) Science 227: 1229-1231

Hudspeth, et al (1991) Plant Physiol. 98: 458-464

Hughes & Galam (1988) Plant Mol. Biol. Rep. 6: 253-257

Isono et al (1997) Proc. Natl. Acad. Sci. USA 94: 14948-14953

Jaffe & Herberman (1988). J Natl. Cancer Inst. 80: 616-618

James, et al (1995) Bio/Technology 13: 592-596

Jefferson et al (1989) EMBO, J. 6: 3901-3907

Karlin & Altschul (1993) Proc. Natl. Acad. Sci. USA 90: 5873-5787

Kestermann et al (1998) Nucleic Acids Res. 26: 2747-2753

Khan & Maliga (1999) Nature Biotech. 17: 910-915

Knoblauch et al (1999) Nature Biotech. 17: 907-909

Krebbers & Vandekerckhove (1990) TIBTECH. 8: 1-3

Ku et al (1999) Nat. Biotechnol. 17: 76-80

Kung & Lin (1985) Nuc. Acids. Res. 13: 7543-7549

Kunkel et al (1999) Nature Biotech. 17: 916-919

Laemmli (1970) Nature 227: 680-685

Lagutin et al (1999) J. Interferon Cytokine Res. 19: 137-144

Lewis (1988) Antiviral activity of cytokines. In: Cytokines, Richwood & Homes (Eds.) Oxford University Press. New York. pp.108-119

Ma et al (1987) Infect Immun. 55: 1274-1278

Ma et al (1995) Science 268: 716-719

Ma et al (1998) Nat. Med. 4: 601-606

Machold et al (1992) Ann. Rheum. Dis. 51: 1039-1043

McBride et al (1995) Bio/Technology 13: 362-365

Mettler I J (1987) Plant Mol. Biol. Rep. 5: 346-349

Miele (1997) TIBTECH 15: 45-50

Murashige & Skoog (1962) Physiol. Plant. 15: 473-497

Nagai et al (l985) PNAS USA 82: 7252-7255

Neuhas et al (1987) Theor. Appl. Genet. 74: 30-36

Okamoto et al (1998) Plant Cell Physiol. 39: 57-63

Perlok et al (1991) Proc. Natl. Acad. Sci. 88: 3324-3328

Pestka et al (1987) Ann. Rev. Biochem. 56: 727-777

Pineiro et al (1999) Plant Cell Rep. 18: 505-508

Potrykus et al (1985) Mol. Gen. Genet. 199: 183-188

Quinlan et al (1989) J. Cell Sci. 93: 71-83

Rinderknecht et al (i 984) J. Biol. Chem. 259: 6790-6797

Rudolph et al (1999) TIBTECH 17: 367-374

Sambrook et al (1989) Molecular Cloning: A Laboratory Manual. Cold Spring Harbor Laboratory Press, Cold Spring Harbor, N.Y.

Saiki et al (1988) Science 239: 487-491

Sarkar & Sommer (1990) Biotechniques 8: 404-407

Schouten et al (1997) FEBS Lett 415: 235-241

Schouten et al (1996) Plant. Mol. Biol. 30: 781-793

Sen & Lengyel (1992) J. Biol. Chem. 267: 5017-5020

Shinozaki et al (1986) EMBO J. 5: 2043-2049

Shinozaki et al (1988) E O J. 5: 2043-2049

Smith & Johnson (1988) Gene 67: 31-40

Sriraman et al (1998a) Plant Physiol. 117: 1495-1499

Sriraman et al (1998b) Nuc. Acids Res. 26: 4874-4879

Staub & Maliga (1992) Plant Cell 4: 39-45

Stuab & Maliga (1993) EMBO J. 12: 601-606

Sugiura (1999) Plant & Cell Physiol. 40: 311-318

Svab (1990) Proc. Natl. Acad. Sci. 87: 8526-8530

Svab & Maliga (1993) Proc. Natl. Acad. Sci. 90: 913-917

Tacket et al (1998) Nat. Med. 4: 607-609

Tanaka et al (1997) FEB. Letters 413: 309-313

Taylor & Walbot (1985) Proc. Natl. Acad. Sci. 82: 5824-5828

Vega et al (1990) Microbiol. Biotechnol. 33: 429-434

Vilcek (1990) Interferons. In: Peptide Growth Factors and their Receptors: Handbook of Experimental Pharmacology, Vol. 95/II, Spom & Roberts (Eds.).

Springer Verlag, Heidelberg: 3-38

Wang et al (1992) International Centre for Genetic Engineering and Biotecchnology, Activity Report, Trieste, New Delhi: 58-60

Wearne (1990) FEBS Lett 263(1): 23-26

Weening et al (1995) Eur. J Pediatr. 154: 295-298

1. A method of obtaining a stable transplastome, which method comprises transforming a recipient plastome with a polynucleotide comprising:
(a) a 5′ sequence homologous to a region of the recipient plastome, and, joined thereto;
(b) a sequence heterologous to the recipient plastome comprising a coding region operably linked to at least one regulatory region capable of securing expression of the coding region in the plastid, and, joined thereto;
(c) a 3′ sequence homologous to a region of the recipient plastome:

2. A method according to claim 1 wherein a regulatory region is derived from a plastomic sequence, or a variant thereof.

3. A method according to claim 1 or 2 wherein the recipient plastome is derived from a multicellular organism.

4. A method according to any one of the preceding claims wherein a regulatory region is derived from the plastome of a monocotyledonous plant.

5. A method according to any one of the preceding claims wherein the recipient plastome is derived from a monocotyledonous plant

6. A method according to any one of claims 1 to 4 wherein the recipient plastome is derived from a dicotyledonous plant with the proviso that:
(a) where the recipient plastome is an Arabidopsis thaliana plastome, the regulatory regions are not derived from the plastome of Nicotiana tabacum; and
(b) where the recipient plastome is an Nicotiana tabacum plastome, the regulatory regions are not derived from the plastome of Spinacia oleracea.

7. A method according to any one of claims 1 to 5 wherein at least one regulatory region is a promoter, enhancer or terminator or a variant thereof.

8. A method according to claim 7 wherein the regulatory region is derived from the plastome of rice.

9. A method according to claim 7 to 8 wherein at least one promoter is the rsn or psbA promoter, or a variant thereof.

10. A method according to claim 7 or 8 wherein at least one regulatory region is a terminator sequence.

11. A method according to claim 10 wherein at least one terminator sequence is the 3′ untranslated region of the psbA or rbcL gene, or a variant thereof.

12. A method of obtaining a stable transplastome, which method comprises transforming a recipient plastome with a polynucleotide comprising:
(a) a 5′ sequence homologous to a region of the recipient plastome, and, joined thereto;
(b) a sequence heterologous to the recipient plastome comprising a coding region, and, joined thereto;
(c) a 3′ sequence homologous to a region of the recipient plastome;
wherein the coding region defined in (b) is operably linked to at least one regulatory region capable of securing expression of the coding region in the plastid, which regulatory region is homologous to a region of the recipient plastid and is positioned in homologous region (a) or (c).

13. A method of obtaining a stable transplastome, which method comprises transforming a recipient plastome of a multicellular organism with a polynucleotide comprising:
(a) a 5′ sequence homologous to a region of the recipient plastome, and, joined thereto;
(b) a sequence heterologous to the recipient plastome comprising a coding region, and, joined thereto;
(c) a 3′ sequence homologous to a region of the recipient plastome;
wherein, following transformation, the coding region defined in (b) is operably linked to at least one regulatory region capable of securing expression of the coding region in the plastid, which regulatory region is an endogenous plastome regulatory region positioned 5′ to homologous region (a) or 3′ to homologous region (c) within the transplatome.

14. A method according to any one of the proceeding claims wherein heterologous region (b) comprises more than one coding region and wherein the coding regions are operably linked to the same or different regulatory regions capable of securing their expression in the transplastome.

15. A method according to any one of the preceding claims wherein at least one homologous region (a) or (c) is derived from the recipient plastome rbcL or accD gene.

16. A method according to any one of claims 7 to 15 wherein the recipient plastome is a tobacco plastome.

17. A method according to any one of the preceding claims wherein the heterologous region of the polynucleotide defined in claim 1(b) comprises at least one coding region encoding a polypeptide able to act as a selectable or scorable marker for the presence of the integrated sequence within the transplastome.

18. A method according to claim 17 wherein a selectable or scorable marker coding region encodes an enzyme capable of acting as an aminoglycodside adenylyltransferase or a β-glucuronidase.

19. A method according to any one of the preceding claims wherein the heterologous region of the polynucleotide defined in claim 1(b) comprises at least one coding region encoding a fusion protein.

20. A method according to claim 19 wherein the fusion protein comprises the amino acid sequence of a polypeptide of interest fused to another amino acid sequence, which other amino acid sequence increases the plastidic accumulation of the expressed fusion protein compared to the plastidic accumulation of the individually expressed polypeptide of interest.

21. A method according to claim 19 or 20 wherein the fusion protein comprises a sequence that has a scorable property.

22. A method according to any one of claims 19 to 21 wherein the fusion protein comprises the sequence of GUS or a variant thereof.

23. A method according to any one of claims 19 to 22 the fusion protein comprises a sequence that enables affinity purification.

24. A method according to claim 23 wherein the sequence is a His-tag or a variant thereof.

25. A method according to any one of claims 19 to 24 wherein the fusion protein comprises one or two amino acid sequences which can be cleaved to release the polypeptide of interest.

26. A method according to claim 25 wherein at least one cleavage sequences is IEGR or a variant thereof.

27. A method according to any one of claims 19 to 26 wherein the fusion protein comprises, as the polypeptide sequence of interest, an amino acid sequence with substantially the same amino acid sequence as a human protein or a variant thereof.

28. A method according to claim 27 wherein the human protein is an interferon or a variant thereof.

29. A method according to claim 28 wherein the interferon is IFN-g or a variant thereof.

30. A method according to claim 29 wherein IFN-g is fused to GUS.

31. A method according to claim 30 wherein IFN-g is fused to GUS via an IEGR cleavage site, and comprises a His-tag.

32. A method of obtaining a transplastomic plastid, which method comprises transforming a plastome with a plastid by a method according to any one of the preceding claims.

33. A method according to claim 32 wherein the plastid is a proplastid, an amyloplast, a chromoplast, a chloroplast, an etioplast or a leucoplast.

34. A method according to claim 33 wherein the plastid is a chloroplast.

35. A method of obtaining a transplastomic cell, which method comprises transforming a plastome within a plastid within a cell by a method according to any one of the preceding claims.

36. A method of obtaining a homotransplastomic cell, which method comprises obtaining transplastomic cells by a method according to claim 35 and selecting for the presence of the transplastome.

37. A method of obtaining a first-generation transplastomic or homotransplastomic plant, wherein the method comprises regenerating a transplastomic or homotransplastomic plant cell obtainable by the method of claim 35 or 36 to give a transplastomic or homotransplastomic plant.

38. A method of obtaining a transplastomic or homotransplastomic plant seed, wherein the method comprises obtaining a transplastomic or homotransplastomic seed from a transplastomic or homotransplastomic plant obtainable by the method of claim 37.

39. A method of obtaining a transplastomic or homotransplastomic progeny plant comprising obtaining a second-generation transplastomic or homotransplastomic progeny plant from a first-generation transplastomic or homotransplastomic plant obtainable by a method according to claim 37, and optionally obtaining transplastomic or homotransplastomic plants of one or more further generations from the second-generation progeny plant thus obtained.

40. A method according to claim 39 comprising:
(a) obtaining a transplastomic or homotransplastomic seed from a first-generation transplastomic or homotransplastomic plant obtainable by the method according to claim 37, then obtaining a second-generation transplastomic or homotransplastomic progeny plant from the seed; and/or
(b) propagating clonally a first-generation transplastomic or homotransplastomic plant obtainable by the method according to claim 37 to give a second-generation transplastomic or homotransplastomic progeny plant; and/or
(c) crossing a first-generation transplastomic or homotransplastomic plant obtainable by a method according to claim 37 with another plant to give a second-generation transplastomic or homotransplastomic progeny plant; and optionally;
(d) obtaining transplastomic or homotransplastomic progeny plants of one or more further generations from the second-generation transplastomic or homotransplastomic progeny plant thus obtained.

41. Use of a polynucleotide as defined in any one of claims 1 to 31, in the production of a stable transplastome, a transplastomic or homotransplastomic plastid, a transplastomic or homotransplastomic cell, a transplastomic or homotransplastomic plant or a transplastomic or homotransplastomic seed.

42. A transplastome, a transplastomic or homotransplastomic plastid, transplastomic or homotransplastomic plant cell, transplastomic or homotransplastomic callus, a transplastomic or homotransplastomic first-generation plant, transplastomic or homotransplastomic plant seed or progeny plant obtainable by a method according to any one of claims 1 to 40.

43. A transplastome transformed with a polynucleotide as defined in any one of claims 1 to 31.

44. A transplastomic or homotransplastomic plastid comprising a transplastome according to claim 43.

45. A transplastomic or homotransplastomic plant cell, callus, first-generation plant, obtainable from a plastid according to claim 44 or a transplastomic or homotransplastomic seed, second-generation progeny plant or a plant of one or more further generations obtainable therefrom.

46. A method of obtaining a crop product comprising harvesting a crop product from a cell or plant obtainable by a method according to any one of claims 35 to 40 or according to claim 42 or 45 and optionally further processing the harvested product.

47. A method of obtaining a crop product according to claim 46 wherein the product is a transplastomically expressed protein and the further processing comprises substantially isolating the protein.

48. A method according to claim 46 wherein the protein is a fusion protein as defined in any one of claims 19 to 31.

49. A method according to claim 48 wherein the protein is a fusion protein as defined in any one of claims 25 to 31, and the further processing also comprises cleavage of the fusion protein, optionally followed by separation of the cleavage products.

50. A crop product obtainable by a method according to any one of claims 46 to 49.

51. Use of a vector for the generation of a stable transplastome, which vector comprises:
(a) a 5′ sequence homologous to a region of the recipient plastome comprising a unique restriction site at its 5′ terminus, and joined at the 3′ terminus by a unique restriction site to;
(b) a sequence heterologous to the recipient plastome comprising:
(i) coding regions-for at least one selectable or scorable marker operably linked to regulatory regions capable of securing expression of the coding sequence in the transplastome; and optionally
(ii) regulatory regions capable of securing expression of a coding sequence in the transplastome, wherein each regulatory region comprises unique restriction sites at 5′ and 3′ borders; and optionally
(iii) a coding sequence operably linked to the regulatory regions of (b)(ii); wherein the 3′ terminus of the heterologous sequence is joined by a unique restriction site to;
(c) a 3′ sequence homologous to a region of the recipient plastome comprising a unique restriction site at its 3′ terminus.

52. Use according to claim 51 wherein the coding sequence as defined in claim 51 (b)(iii) encodes a polypeptide comprising the amino acid sequence of GUS, or a biologically active variant thereof.
